(19) United States
(12) Patent Application Publication
(10) Pub. No.: US 2012/0095510 A1
(43) Pub. Date: Apr. 19, 2012

(54) CROSS CONNECTORS

(76) Inventor: Raj Nihalani, Irvine, CA (US)

(21) Appl. No.: 12/906,991

(22) Filed: Oct. 18, 2010

(51) Int. Cl. A61B 17/70

(52) U.S. Cl. 606/250; 606/264

(57) ABSTRACT

The present invention may provide various improvements over conventional cross connectors. For example, the present invention may provide various types of Real-X cross connectors, which may have an arch shape X-bridge that curves above the spinal bone segments of the patient. As such, the Real-X cross connectors may be more adaptive to the patient's spinal provide and provide better protect for the patient's the spinal bone segments. For another example, the present invention may provide various types of Real-O cross connectors, which may have a protection ring for protecting the patient's spinous process. Because of its protection ring, the implantation of one of the Real-O cross connectors may eliminate the need of spinous process removal. Furthermore, a Real-O cross connector may be combined with a Real-X cross connector to form a Real-XO cross connector, which may inherit the functional benefits of both the Real-X and Real-O cross connectors.

### BACKGROUND

1. Field

The present invention relates generally to the field of medical devices used in posterior spinal fixation surgery, and more particularly to cross connectors.

2. Description of the Related Art

Posterior spinal fixation surgery is a common procedure for patients who suffer from severe spinal conditions, such as spinal displacement, spinal instability, spinal degeneration, and/or spinal stenosis. Among other therapeutic goals, a successful posterior spinal fixation surgery may lead to the stabilization and fusion of several spinal bone segments of a patient. During a posterior spinal fixation surgery, a spine surgeon may insert several pedicle screws into one side of several spinal bone segments of the patient to establish several anchoring points. Then, the spine surgeon may engage and secure a stabilizing rod to the several anchoring points to restrict or limit the relative movement of the spinal bone segments.

Next, this procedure may be repeated on the other side of the spinal bone segments, such that two stabilizing rods may be anchored to both sides of the spinal bone segments of the patient. To further restrict or limit the relative movement of the spinal bone segments, a connector may be used to connect the two stabilizing rods, so that the two stabilizing rods may maintain a relatively constant distance from each other. When the posterior spinal fixation surgery is completed, the operated spinal bone segments may be substantially stabilized such that they may be in condition for spinal fusion.

Conventional connectors may suffer from several drawbacks. For example, some conventional connectors may be made of flat and straight arms, such that surgeons may have a difficult time in adjusting these connectors to fit the contour the of patient's spinal bone segments. Accordingly, the implantation of these conventional connectors may require the removal of the patient's spinous process from one or more spinal bone segments because they may not be adaptive to the spinal bone structure of the patient. Moreover, most conventional connectors may not be able to protect any damaged spinal bone segment of the patient because they are formed by a thin strip of alloy, which can only cover a small area. Furthermore, most conventional connectors lack pre-fixation flexibility, such that they may not be adjusted to fit patients with various spinal bone widths or asymmetrical spinal bone profile.

Thus, there are needs to provide cross connectors with improved features and qualities.

### SUMMARY

The present invention may provide various improvements over conventional connectors. For example, the present invention may provide various types of Real-X cross connectors, which may have an arch shape X-bridge that curves above the spinal bone segments of the patient. As such, the Real-X cross connectors may be more adaptive to the patient's spinal bone contour and provide better protect for the patient's spinal bone segments. For another example, the present invention may provide various types of Real-O cross connectors, which may have a protection ring that may surround the patient's spinous process. Because of its protection ring, the implantation of one of the Real-O cross connectors may eliminate the need of spinous process removal. Furthermore, as provided by the present invention, the Real-O cross connector may be combined with the Real-X cross connector to form a Real-XO cross connector, which may inherit the functional benefits of both Real-X and Real-O cross connectors.

In one embodiment, the present invention may provide a cross connector for use in conjunction with four or more pedicle screws for stabilizing and protecting one or more fixation levels of spinal bone segments. The cross connector may be configured to be anchored to the spinal bone segments by four or more pedicle screws, and it may include first and second elongated members each having first and second ends and a pivot segment positioned between the first and second ends, a fulcrum member configured to engage the pivot segment of the first elongated member and the pivot segment of the second elongated member, thereby allowing a relative movement therebetween, and a plurality of connecting devices, each configured to connect one of the first end or the second end of one of the first elongated stabilizer or the second elongated stabilizer to one of the four or more pedicle screws, such that the first and second elongated members are configured to form an X-shape bridge across the one or more fixation levels of spinal bone segments.

In another embodiment, the present invention may provide a cross connector for use in conjunction with first and second stabilizing rods for stabilizing and protecting one or more fixation levels of spinal bone segments. The first and second stabilizing rods may be configured to be anchored to left and right pedicles of the spinal bone segments. The cross connector is configured to be anchored to the spinal bone segments via the first and second stabilizing rods, and it may include first and second elongated members each having first and second ends and a pivot segment positioned between the first and second ends, a fulcrum member configured to engage the pivot segment of the first elongated member and the pivot segment of the second elongated member, thereby allowing a relative movement therebetween, a first anchoring device anchoring the first end of the first elongated member to the first stabilizing rod, a second anchoring device anchoring the second end of the first elongated member to the second stabilizing rod, a third anchoring device anchoring the first end of the second elongated member to the second stabilizing rod, and a fourth anchoring device anchoring the second end of the second elongated member to the first stabilizing rod, such that the first and second elongated members are configured to form an X-shape bridge across the one or more fixation levels of spinal bone segments.

In another embodiment, the present invention may include a cross connector for use in conjunction with first and second stabilizing rods for stabilizing and protecting one or more fixation levels of spinal bone segments. The first and second stabilizing rods may be configured to be anchored to left and right pedicles of the spinal bone segments. The cross connector may be configured to be anchored to the spinal bone segments via the first and second stabilizing rods, and it may include a first arm configured to be anchored to the first stabilizing rod, a center member having first and second ends and a pair of brackets joining the first and second ends to form a protection ring, the first end coupled to the first arm, the protection ring configured to laterally surround a spinous process of one of the spinal bone segment, and a second arm coupled to the second end of the center member and configured to be anchored to the second stabilizing rod.

In another embodiment, the present invention may provide a cross connector which may include a ring member having a circumferential surface, first and second arms, each of the first and second arms having first and second ends, the first ends of the first and second arms configured to be coupled to the circumferential surface of the ring member, such that the first and second arms form a first arched bridge for supporting the ring member, and first and second connecting devices, the first connecting device configured to be coupled to the second end of the first arm, the second connecting device configured to be coupled to the second end of the second arm.

In yet another embodiment, the present invention may provide a lockable joint for coupling a connecting device to an end of a cross connector. The lockable joint may include a housing having a top surface, a side wall, an inner socket surface, the top receiving port formed on the top surface, a side receiving port formed on the side wall, the side wall configured to be coupled to the connecting device, a bearing disposed within the housing and contacting the inner socket surface of the housing, a handle coupled to the bearing, the handle configured to extend outside the housing via the side opening, and configured to be coupled to the end of the cross connector, such that the handle has a range of multi-axle movement about the bearing, and a locking screw having a concave surface, the locking screw configured to engage the housing via the top receiving port, the concave surface configured to apply a compression force against the bearing when the locking screw is at a locking position, the compression force substantially restricting the range of multi-axle movement of the handle.

### BRIEF DESCRIPTION OF THE DRAWINGS

Other systems, methods, features, and advantages of the present invention will be or will become apparent to one skilled in the art upon examination of the following figures and detailed description. It is intended that all such additional systems, methods, features, and advantages be included within this description, be within the scope of the present invention, and be protected by the accompanying claims. Component parts shown in the drawings are not necessarily to scale, and may be exaggerated to better illustrate the important features of the present invention. In the drawings, like reference numerals designate like parts throughout the different views, wherein:

FIGS. 1A-1C show various views of a Real-X cross connector according to an embodiment of the present invention;

FIGS. 1D-1G show various views of the Real-X cross connector being anchored to three spinal bone segments according to an embodiment of the present invention;

FIGS. 2A-2C show various views of a Real-X cross connector with four anchoring devices according to an embodiment of the present invention;

FIGS. 2D-2F show a top perspective view and the top views of the Real-X cross connector with four hook members being anchored to three spinal bone segments according to an embodiment of the present invention;

FIGS. 3A-3C show various views of a Real-X cross connector with four articulated rods as the connecting devices according to an embodiment of the present invention;

FIGS. 3D-3H show a top perspective view and the top views of the Real-X cross connector with four articulated rods being anchored to three spinal bone segments according to an embodiment of the present invention;

FIGS. 4A-4C show various views of a Real-X cross connector with adjustable arms according to an embodiment of the present invention;

FIGS. 4D-4F show the cross-sectional side views of several configurations of the arm length adjustable device according to various embodiments of the present invention;

FIGS. 4G-4I show various configurations of the Real-X cross connector with the adjustable arms according to various embodiments of the present invention;

FIGS. 5A-5C show various views of a fulcrum member according to an embodiment of the present invention;

FIGS. 6A-6C show various views of an alternative fulcrum member according to an alternative embodiment of the present invention;

FIGS. 7A-7C show various views of a Real-X cross connector with two adjustable rods as the connecting devices according to an embodiment of the present invention;

FIGS. 8A-8B show a perspective view and a cross-sectional side view a Real-O cross connector (ROCC) according to an embodiment of the present invention;

FIGS. 8C-8D show a perspective view and a cross sectional side view of an alternative Real-O cross connector (ROCC) according to another embodiment of the present invention;

FIG. 8E shows a top view of the ROCC being anchored between two stabilizing rods according to an embodiment of the present invention;

FIGS. 8F-8G show the top views of the alternative ROCC being anchored between two stabilizing rods according to an embodiment of the present invention;

FIGS. 9A-9B show a perspective view and a cross-sectional side view of a Real-O cross connector with an adjustable ring according to an embodiment of the present invention;

FIGS. 10A-10H show the Real-O cross connector with rings of various shapes according to various embodiments of the present invention;

FIGS. 11A-11D show various views of a Real-XO cross connector(RXOCC) according to an embodiment of the present invention;

FIGS. 11E-11G show various configurations of the RXOCC according to various embodiments of the present invention; and

FIGS. 12A-12E show various views of an alternative lockable joint member according to an alternative embodiment of the present invention.

### DETAILED DESCRIPTION

Apparatus, systems and methods that implement the embodiment of the various features of the present invention will now be described with reference to the drawings. The drawings and the associated descriptions are provided to illustrate some embodiments of the present invention and not to limit the scope of the present invention. Throughout the drawings, reference numbers are re-used to indicate correspondence between reference elements. In addition, the first digit of each reference number indicates the figure in which the element first appears.

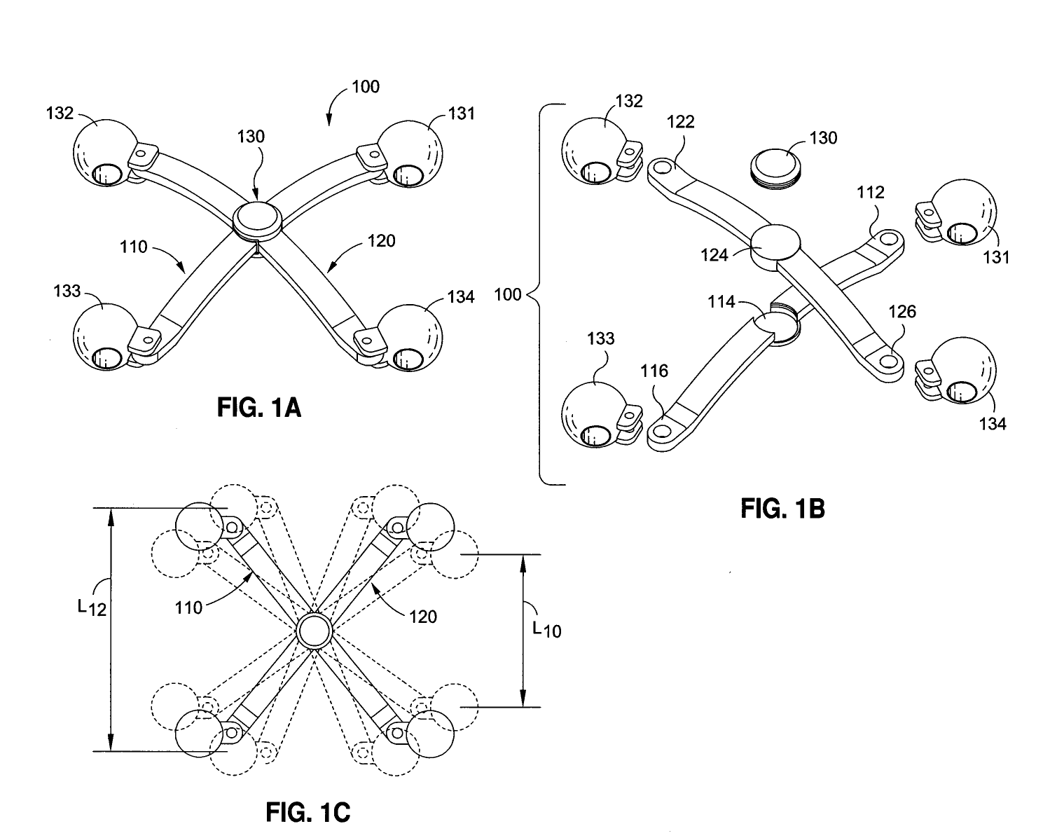

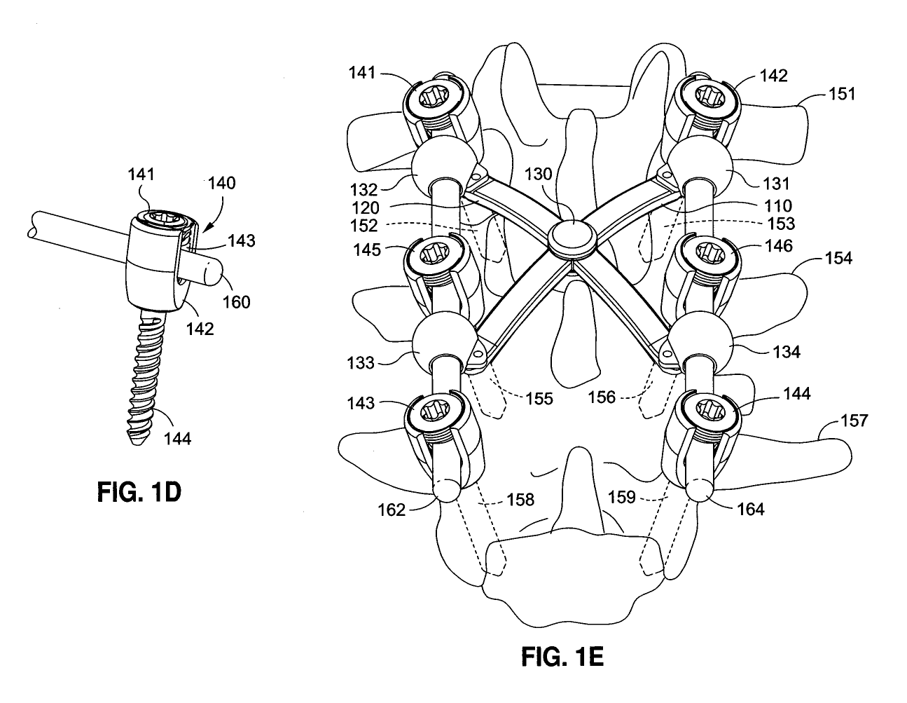

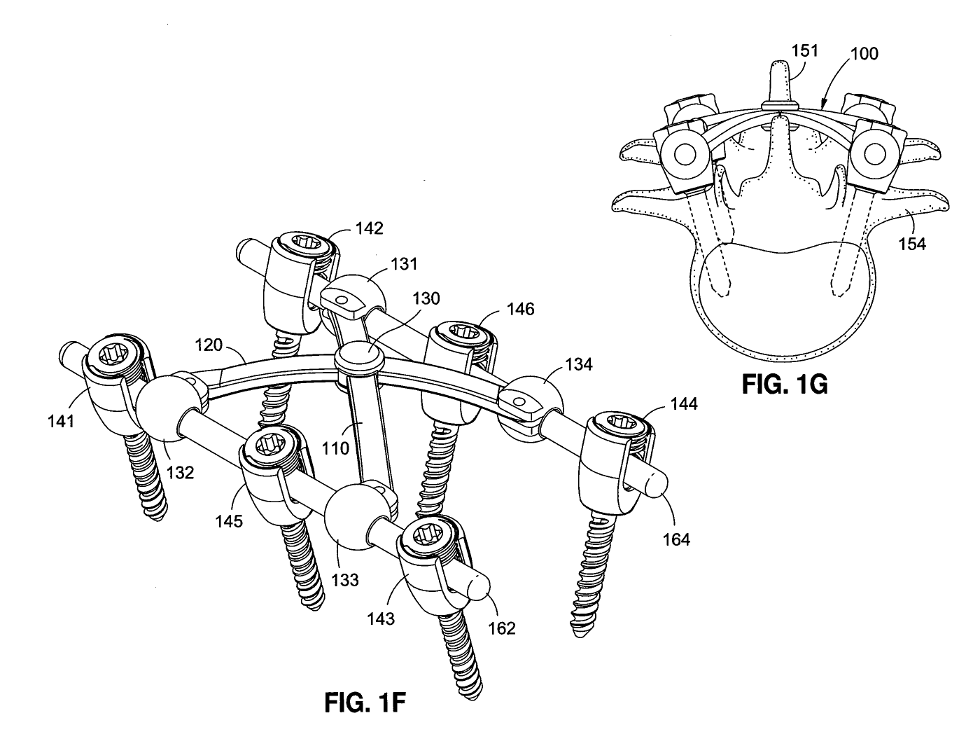

FIGS. 1A-1C show various views of a Real-X cross connector (RXCC) 100 according to an embodiment of the present invention. As shown in FIG. 1A, the RXCC 100 may include a first elongated member (first arm) 110, a second elongated member (second arm) 120, a fulcrum member 130, and four connecting devices 131, 132, 133, and 134. Generally, as shown in FIG. 1B, the first and second elongated members 110 and 120 may have first ends 112 and 122, second ends 116 and 126, and pivot segments 114 and 124.

In one embodiment of the present invention, the fulcrum member 130 may engage both the pivot segment 114 of the first elongated member 110 and the pivot segment 124 of the second elongated member 120. Consequently, as shown in FIG. 1C, the first elongated member 110 may have a range of pivotal movement with the second elongated member 120. Advantageously, the RXCC 100 may be adjusted to have a minimum width L₁₀ and a maximum width L₁₂ between the first ends 112 and 122 and/or the second ends 116 and 126. In one embodiment, the minimum width L₁₀ may be about 5 mm while the maximum width L₁₂ may be about 120 mm. In another embodiment, the minimum width L₁₀ may be about 10 mm while the maximum width L₁₂ may be about 100 mm. In yet another embodiment, the minimum width L₁₀ may be about 12 mm while the maximum width L₁₂ may be about 88 mm.

As shown in FIG. 1B, the first and second elongated members 110 and 120 may each have an arch. In one embodiment, the pivot segments 114 and 124 may form the top parts of the arch, whereas the first and second ends 112, 122, 116, and 126 may form the bottom parts of the arch. Together, the first and second elongated members 110 and 120 may form an X-shape protection bridge with a convex profile, which may fit and adapt to a posterior contour of several spinal bone segments. Advantageously, the RXCC 100 may be placed across one or more spinal bone segments for protecting a defected bone segment or a partially exposed spinal cord (not shown).

Moreover, the RXCC 100 may be equipped with the first connecting device 131, the second connecting device 132, the third connecting device 133, and the fourth connecting device 134. More specifically, the first connecting device 131 may be coupled to the first end 112 of the first elongated member 110, the second connecting device 132 may be coupled to the first end 122 of the second elongated member 120, the third connecting device 133 may be coupled to the second end 116 of the first elongated member 110, and the fourth connecting device 134 may be coupled to the second end 126 of the second elongated member 120.

The four connecting devices 131, 132, 133, and 134 may be used for connecting the RXCC 100 to a group of pedicle screws or two stabilizing rods, both of which may be anchored to one or more spinal bone segments. As such, the RXCC 100 may substantially reduce or minimize the relative movement among the pedicle screws or among the two stabilizing rods. Advantageously, the RXCC 100 may provide extra support and stability to one or more spinal bone segments by virtue of connecting to the group of pedicle screws or the two stabilizing rods.

FIGS. 1D-1F show various views of the Real-X cross connector (RXCC) 100 being anchored to three spinal bone segments 151, 154, and 157 according to an embodiment of the present invention. Generally, as shown in FIG. 1D, a pedicle screw 140 may include a set screw 141, a threaded shaft 144, and a base member 142. More specifically, the threaded shaft 144 may be used for drilling into a spinal bone segment, the base member 142 may have a pair of receiving ports 143 for receiving a stabilizing rod 160, and the set screw 141 may be used for securing the stabilizing rod 160 to the base member 142.

Referring to FIG. 1E, six pedicle screws 141, 142, 143, 144, 145, and 146 may be used to anchor the spinal bone segments 151, 154, 157. For example, the pedicle screws 141 and 142 may be drilled into the spinal bone segments 151 via the left pedicle 152 and the right pedicle 153 respectively. For another example, the pedicle screws 145 and 146 may be drilled into the spinal bone segments 154 via the left pedicle 155 and the right pedicle 156 respectively. For yet anther example, the pedicle screws 143 and 144 may be drilled into the spinal bone segments 157 via the left pedicle 158 and the right pedicle 159 respectively.

After the anchoring process, the first stabilizing rod 162 may be received and secured by the anchored pedicle screws 141, 143, and 145, while the second stabilizing rod 164 may be received and secured by the anchored pedicle screws 142, 144, and 146. Accordingly, the first stabilizing rod 162 may be anchored to the spinal bone segments 151, 154, and 157 along a left pedicle line defined by the left pedicles 152, 155, and 158, and the second stabilizing rod 164 may be anchored to the spinal bone segments 151, 154, and 157 along a right pedicle line defined by the right pedicles 153, 156, and 159. Depending on the particular group of spinal bone segments being operated on, the left and right pedicle lines may be parallel to each other or they may be angularly positioned.

Next, the RXCC 100 may be placed over the spinal bone segments 151, 154, and 157. For example, as shown in FIGS. 1E and 1F, the first connecting member 131 may connect the first end 112 of the first elongated member 110 to the second stabilizing rod 164 between the pedicle screws 142 and 146, the second connecting member 132 may connect the first end 122 of the second elongated member 120 to the first stabilizing rod 162 between the pedicle screws 141 and 145, the third connecting member 133 may connect the second end 126 of the second elongated member 120 to the second stabilizing rod 164 between the pedicle screws 146 and 144, and the fourth connecting member 134 may connect the second end 116 of the first elongated member 110 to the first stabilizing rod 161 between the pedicle screws 145 and 143.

After the RXCC 100 is connected to the first and second stabilizing rods 162 and 164, the RXCC 100 may form the X-shape protection bridge over and across one or more spinal bone segments. In one configuration, the RXCC 100 may form the X-shape protection bridge for protecting the spinal bone segment 154. In another configuration, the RXCC 100 may form the X-shape protection bridge for protecting the spinal bone segment 151. In yet another configuration, the RXCC 100 may form the X-shape protection bridge for protecting the spinal bone segment 151.

Advantageously, because the first and second elongated members 110 and 120 may have the range of relative pivotal movement as shown in FIG. 1C, the RXCC 100 may be adjusted to adapt to spinal bone segments with various widths. Moreover, as shown in FIGS. 1F and 1G, the convex profile of the X-shape protection bridge may arch over the bone protrusions of one or more spinal bone segments, such that no additional surgical procedure may be require to remove any of these bone protrusions. Furthermore, the RXCC 100 may further stabilize the spinal bone segments 151, 154 and 157 by restricting and/or limiting a relative movement between the first and second stabilizing rods 162 and 164.

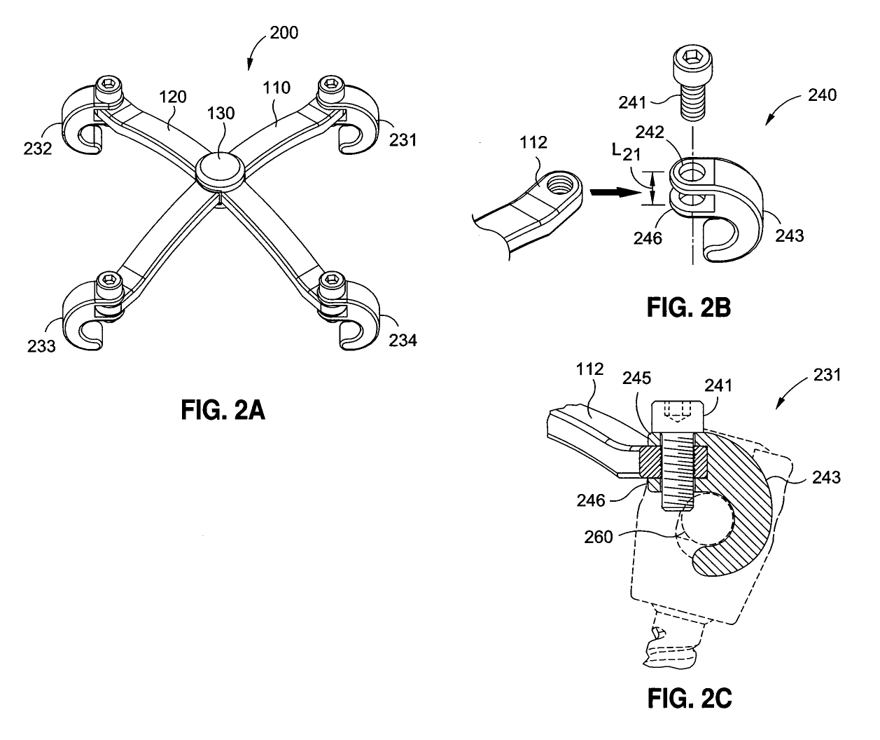

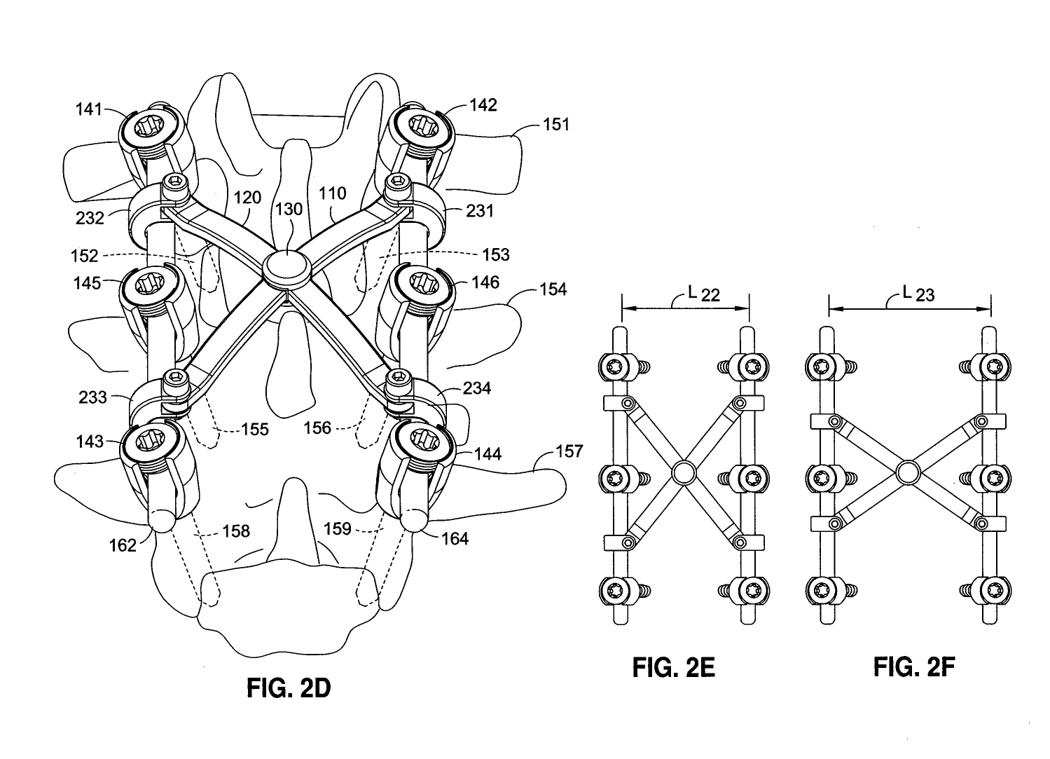

According to an embodiment of the present invention, FIGS. 2A-2C show various views of a Real-X cross connector (RXCC) 200 with four anchoring devices 231, 232, 233, and 234. The RXCC 200 may be similar to the RXCC 100 in several aspects. For example, the RXCC 200 may include the first elongated member (first arm) 110, the second elongated member (second arm) 120, and the fulcrum member 130. For another example, the first and second elongated members 110 and 120 may have first ends 112 and 122, second ends 116 and 126, and pivot segments 114 and 124. For yet another example, RXCC 200 may form an X-shape protection bridge, which may have similar structural and functional features as the X-shape protection bridge of the RXCC 100.

Despite these similarities, the RXCC 200 may be different from the RXCC 100 in at least one embodiment. For example, the RXCC 200 may incorporate four anchoring devices 231, 232, 233, and 234 to perform the functions of the connecting devices 131, 132, 133, and 134 of the RXCC 100 as shown in FIGS. 1A-1F. According to an embodiment of the present invention, the four anchoring devices 231, 232, 233, and 234 may share the structural and functional features of an anchoring device 240 as shown in FIG. 2B.

Generally, the anchoring device 240 may include a locking screw 241, a joint member 242, and a hook member 243. More specifically, the joint member 242 may be attached to the hook member 243 while the locking screw 241 may be a separate structure. The joint member 242 may have a first disc member 245, a second disc member 246, and a space defined therebetween. In order to properly receive one of the first ends 112 or 122 or one of the second ends 116 or 126, the space may have a height L₂₁, which may be slightly greater than the thickness of each of the first and second ends 112, 122, 116, and 126. Moreover, in order to properly receive the locking screw 241, both the first and second discs 245 and 246 may each have an opening with a diameter slightly greater than a diameter of the locking screw 241.

Referring to FIG. 2C, which shows the operation of the anchoring device 231, the first end 112 of the first elongated member 110 may be inserted into the space between the first and second disc members 245 and 246 of the joint member 242, and the hook member 243 may engage a segment of a stabilizing rod 260. Next, the locking screw 241 may penetrate the first and second disc members 245 and 246 as well as the first end 112 received therebetween. Consequentially, the first end 112 may be secured to the anchoring device 231 and it may freely rotate about the locking screw 241.

In order to limit the movement of the first end 112 in relative the anchoring device 231, the locking screw 241 may fully engage the first and second disc members 245 and 246. The locking screw 241 may cooperate with the first and second disc members 245 and 246 to assert a pair of vertical forces against the top and bottom surfaces of the first end 112. Accordingly, the friction between the joint member 242 and the first end 112 may increase substantially, and the relative movement of the first end 112 may be locked at a particular angular position in relative to the hook member 243.

The above assembling procedures may be repeated for the first end 122 of the second elongated member 120, the second end 116 of the first elongated member 110, and the second end 126 of the second elongated member 120. Accordingly, the first anchoring device 231 may be coupled to the first end 112, the second anchoring device 232 may be coupled to the first end 122, the third anchoring device 233 may be coupled to the second end 116, and the fourth anchoring device 234 may be coupled to the second end 126.

After the initial assembling process, the hook member 243 may be used to engage a segment of the stabilizing rod 260. When the anchoring device is properly positioned, the locking screw 241 may be driven further to contact the segment of the stabilizing rod 260. In one embodiment of the present invention, the locking screw 241 may assert a compression force against a top part of the stabilizing rod 260, which may redirect the compression force against a bottom section of the hook member 243. As a result, the bottom section of the hook member 243 may react to the compression force and produce a reaction force, which may be asserted against a bottom part of the stabilizing rod 260. Accordingly, the compression force may cooperate with the reaction force to secure the segment of stabilizing rod 260 within the hook member 243.

FIG. 2D shows a top perspective view of the RXCC 200 anchored to three spinal bone segments 151, 154, and 157 via the pedicle screws 141, 142, 143, 144, 145, and 146 and the stabilizing rods 162 and 164. Generally, the pedicle screws 141, 142, 143, 144, 145, and 146 and the stabilizing rods 162 and 164 may be first anchored to the left and right pedicles of the spinal bone segment 151, 154, and 157 as discussed in FIGS. 1E and 1F. Like the RXCC 100, the RXCC 200 may form the X-shape protection bridge above and across the spinal bone segment 151, 154, or 157.

For example, to form the X-shape protection bridge above and across the spinal bone segment 154, the anchoring device 231 may engage the first stabilizing rod 162 between the pedicle screws 141 and 145, the anchoring device 234 may engage first stabilizing rod 162 between the pedicle screws 145 and 143, the anchoring device 232 may engage the second stabilizing rod 164 between the pedicle screws 142 and 146, and the anchoring device 233 may engage the second stabilizing rod 164 between the pedicle screws 146 and 144.

At this stage, the respective locking screws 241 may be free from contacting the first and second stabilizing rods 162 and 164, such that the RXCC 200 may still be free to slide along the first and second stabilizing rods 162 and 164. Advantageously, the X-shape protection bridge may be conveniently maneuvered to cover an area which may need to be protected. After the X-shape protection bridge is properly positioned, the respective locking screws 241 may be applied to secure the first and second rods 162 and 164 to the RXCC 200. Consequentially, the RXCC 200 may be anchored to the first and second rods 162 and 164 via the anchoring devices 231, 232, 233, and 235. At this stage, the RXCC 200 may remain relatively stationary with respect to the first and second stabilizing rods 162 and 164, the pedicle screws 141, 142, 143, 144, 145, and 146, and the spinal bone segments 151, 154, and 157.

As shown in FIGS. 2E and 2F, the RXCC 200 may be adjusted to adapt to spinal bone segments with various width. In one configuration, the RXCC 200 may be adjusted to reduce the distance between the first ends 112 and 122 or between the second ends 116 and 126 if the spinal bone segments 282 have a narrow width L₂₂. Accordingly, the first and second anchoring devices 231 and 232 may be positioned closer to the pedicle screws 141 and 142, while the third and fourth anchoring devices 233 and 234 may be positioned closer to the pedicle screws 143 and 144. In another configuration, the RXCC 200 may be adjusted to increase the distance between the first ends 112 and 122 or between the second ends 116 and 126 if the spinal bone segments 283 have a wide width L₂₃. Accordingly, the first and second anchoring devices 231 and 232 may be positioned farther away from the pedicle screws 141 and 142, while the third and fourth anchoring devices 233 and 234 may be positioned farther away from the pedicle screws 143 and 144.

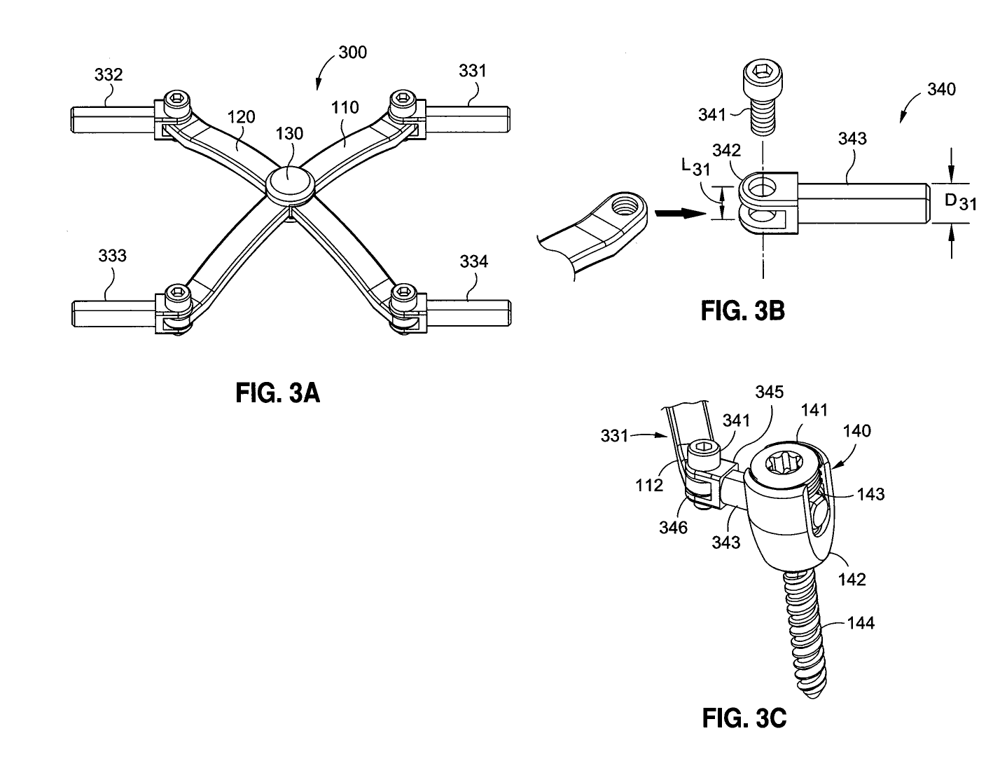

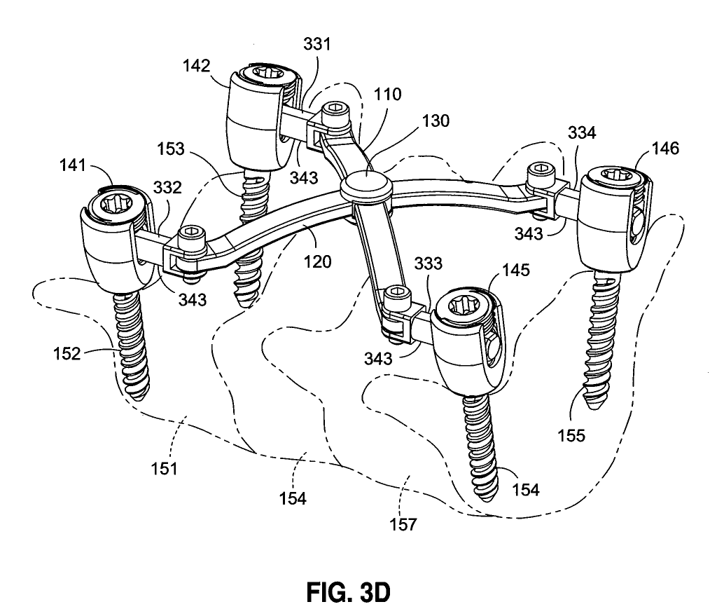

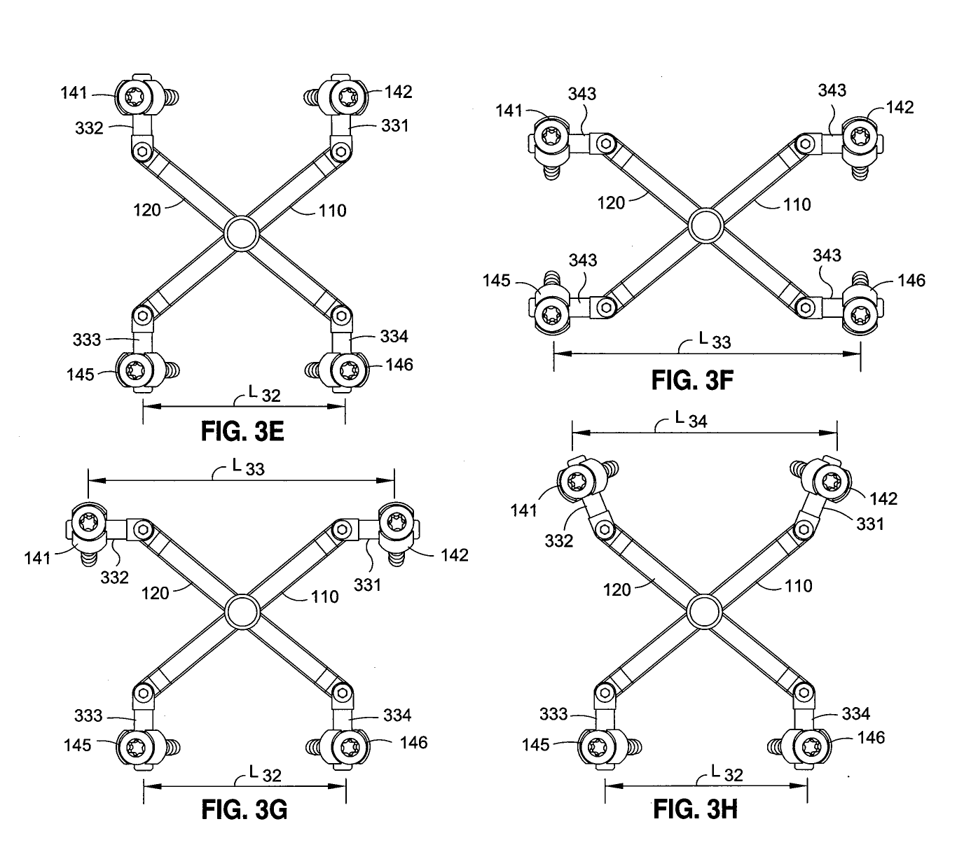

FIGS. 3A-3C show various views of a Real-X cross connector (RXCC) 300 with four articulated rods 331, 332, 333, and 334. The RXCC 300 may be similar to the RXCC 100 in several aspects. For example, the RXCC 300 may include the first elongated member (first arm) 110, the second elongated member (second arm) 120, and the fulcrum member 130. For another example, the first and second elongated members 110 and 120 may have first ends 112 and 122, second ends 116 and 126, and pivot segments 114 and 124. For yet another example, the RXCC 300 may form an X-shape protection bridge, which may have similar structural and functional features as the X-shape protection bridge formed by the RXCC 100.

Despite these similarities, the RXCC 300 may be different from the RXCC 100 in at least one aspect. For example, the RXCC 300 may incorporate four articulated rods 331, 332, 333, and 334 to perform the functions of the connecting devices 131, 132, 133, and 134 of the RXCC 100 as shown in FIGS. 1A-1F. The four articulated rods 331, 332, 333, and 334 may share the structural and functional features of an articulated rod 340 as shown in FIG. 3B.

Generally, the articulated rod 340 may include a locking screw 341, a joint member 342, and a rod member 343. More specifically, the joint member 342 may be attached to the rod member 343 while the locking screw 341 may be a separate structure. The joint member 342 may have a first disc member 345, a second disc member 346, and a space defined therebetween. In order to properly receive one of the first ends 112 or 122 or one of the second ends 116 or 126, the space may have a height L₃₁ slightly greater than the thickness of each of the first and second ends 112, 122, 116, and 126. Moreover, in order to properly receive the locking screw 341, both the first and second discs 345 and 346 may each have an opening with a diameter slightly greater than a diameter of the locking screw 341.

Referring to FIG. 3C, which shows the operation of the articulated rod 331, the first end 112 of the first elongated member 110 may be inserted into the space between the first and second disc members 345 and 346 of the joint member 342, and the rod member 343 may be secured by the pedicle screw 140. Next, the locking screw 341 may penetrate the first and second disc members 345 and 346 as well as the first end 112 positioned therebetween. Consequentially, the first end 112 may be secured to the articulated rod 331 and it may freely rotate about the locking screw 341.

In order to limit the movement of the first end 112 in relative the anchoring device 331, the locking screw 341 may fully engage the first and second disc members 345 and 346. The locking screw 341 may cooperate with the first and second disc members 345 and 346 to assert a pair of vertical forces against the surfaces of the first end 112. As such, the friction between the first and second disc members 345 and 346 and the first end 312 may increase significantly, and the relative movement of the first end 112 may thus be substantially reduced or limited.

The above assembling procedures may be repeated for the first end 122 of the second elongated member 120, the second end 116 of the first elongated member 110, and the second end 126 of the second elongated member 120. Accordingly, the first articulated rod 331 may be coupled to the first end 112, the second articulated rod 332 may be coupled to the first end 122, the third articulated rod 333 may be coupled to the second end 116, and the fourth articulated rod 334 may be coupled to the second end 126.

After the initial assembling process, the rod member 343 may be received by and secured to the pedicle screw 140, which may include components as previously shown in FIG. 1D. For example, the pedicle screw 140 may have the set screw 141, the base member 142 with the pair of receiving ports 143, and the threaded shaft 144 for drilling the spinal bone segment. Initially, the rod member 343 may be inserted into the receiving ports 143 of the pedicle screw 140. When coupled to the base member 142, the set screw 141 may apply a compression force against a top part of the rod member 343, which may redirect the compression force to the base member 142. In reacting to the compression force, the base member 142 may assert a reaction force against a bottom part of the rod member 343. As such, the reaction force may cooperate with the compression force to secure a segment of the rod member 343 to the pedicle screw 140.

The rod member 343 may have similar structural and physical properties as the conventional stabilizing rods 162 and 164 as previously shown and discussed in FIGS. 1D-1F and in FIGS. 2D-2F. Accordingly, the rod member 343 may be made of a similar material as the conventional stabilizing rods 162 and 164, and it may have a diameter D₃₁ similar to those of the conventional stabilizing rods 162 and 164. Nevertheless, the rod member 343 may be substantially shorter than the convention stabilizing rods 162 and 164 because it may only be required to extend for a relatively shorter distance. Moreover, the rod member 343 may have a flat top surface and a flat bottom surface, such that it may be secured by the pedicle screw 140 more efficiently.

FIG. 3D shows a top perspective view of the RXCC 300 anchored to three spinal bone segments 151, 154, and 157 via the pedicle screws 141, 142, 143, and 144. According to an embodiment of the present invention, the RXCC 300, when equipped with the several articulated rods 331, 332, 333, and 334, may provide similar functions as the conventional stabilizing rods 162 and 164 as previously shown in FIGS. 1A-1F and 2A-2F. For example, the first and second elongated members 110 and 120 may substantially reduce the relative movement among the spinal bone segments 151, 154, and 157 when the articulated rods 331, 331, 333, and 334 are properly anchored to the spinal bone segments 151 and 157 via the pedicle screws 141, 142, 143, and 144. Because the RXCC 300 may extend vertically and horizontally, it may provide both vertical and horizontal stabilizations to the spinal bone segments 151, 154, and 157. Advantageously, this bidirectional stabilization substantially improves the unidirectional stabilization provided by the conventional stabilizing rods 162 and 164 because it may better address the horizontal instability among several spinal bone segments.

Moreover, the RXCC 300 may obviate the need for applying the pedicle screws 145 and 146 to the spinal bone segment 154. Furthermore, the RXCC 300 may be applied to two or more fixation levels of spinal bone segments. Accordingly, the RXCC 300 may reduce the number of implantable devices and the number of procedures for installing these implantable devices. Advantageously, using the RXCC 300 may help reduce the cost and time for performing posterior spinal surgery, thereby rendering it more affordable for the patients and more efficient for the surgeons.

FIGS. 3E-3H show various configurations of the RXCC 300 according to various embodiments of the present invention. Similar to the RXCC 100 and the RXCC 200, the RXCC 300 may be adjustable to adapt to spinal bone segments with various widths. Moreover, the extra length and maneuverability provided by the articulated rods 331, 332, 333, and 334 may allow the RXCC 300 to have a wider range of adjustment.

In one embodiment, for example, the RXCC 300 may be adjusted to adapt to the spinal bone segments 381 with a small width L₃₂ as shown in FIG. 3E. In another embodiment, for example, the RXCC 300 may be adjusted to adapt to the spinal bone segments 382 with a large width L₃₃ as shown in FIG. 3F. In another embodiment, for example, the RXCC 300 may be adjusted to adapt to the spinal bone segments 383 with a large top width L₃₃ but a small bottom width L₃₂ as shown in FIG. 3G. Particularly, the rod members 343 of the first and second articulated rods 331 and 332 may be positioned horizontally while the rod members 343 of the third and fourth articulated rods 333 and 334 may be positioned vertically. In yet another embodiment, for example, the RXCC 300 may be adjusted to adapt to the spinal bone segments 384 with a medium top width L₃₄ and a small bottom width L₃₂ as shown in FIG. 3H. Particularly, the rod members 343 of the first and second articulated rods 331 and 332 may positioned diagonally while the third and fourth articulated rods 333 and 334 may be positioned vertically.

Besides the configurations as shown in FIGS. 3E-3F, the RXCC 300 may be adjusted to adapt to a wide range of symmetrical spinal bone segments as well as asymmetrical spinal bone segments. The rod members 343 may be highly maneuverable about the respective joint members 342, and thus, they can be configured to turn in any planar direction before they are firmly secured by the respective pedicle screws 140. Advantageously, the RXCC 300 may provide a dynamic range of configurations, which may be more adjustable and adaptable than the configurations provided by conventional cross connectors and the conventional stabilizing rods.

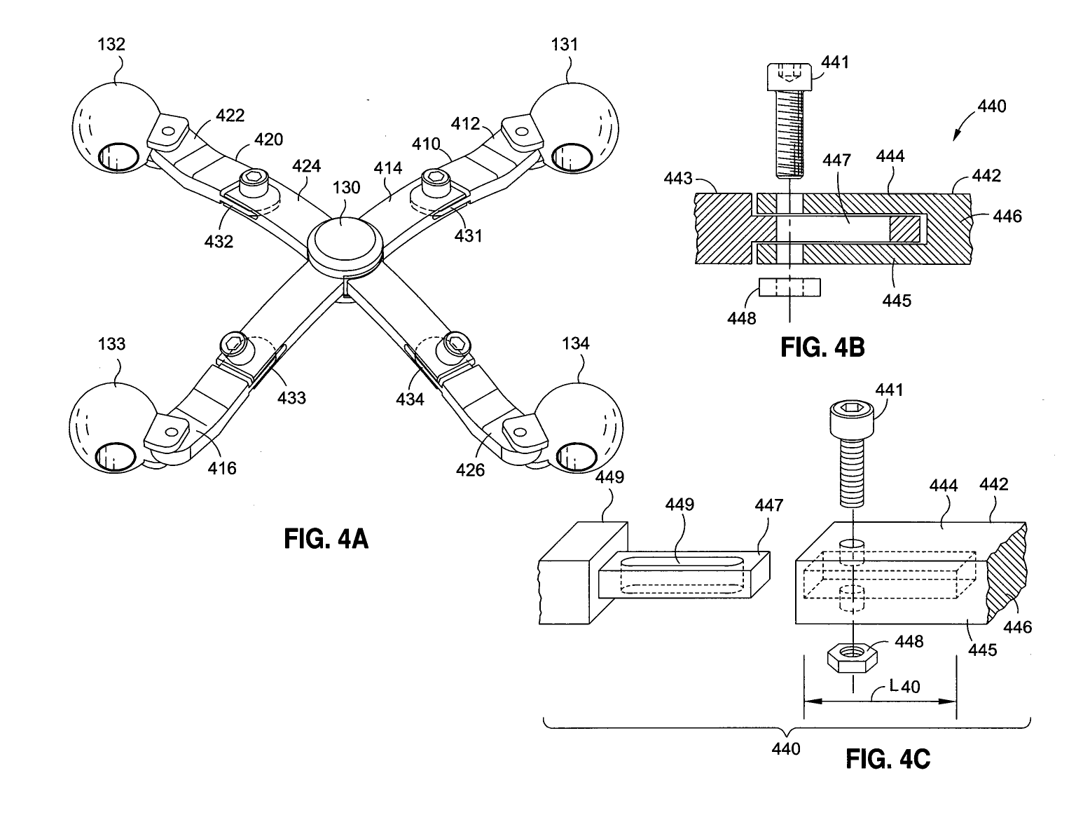

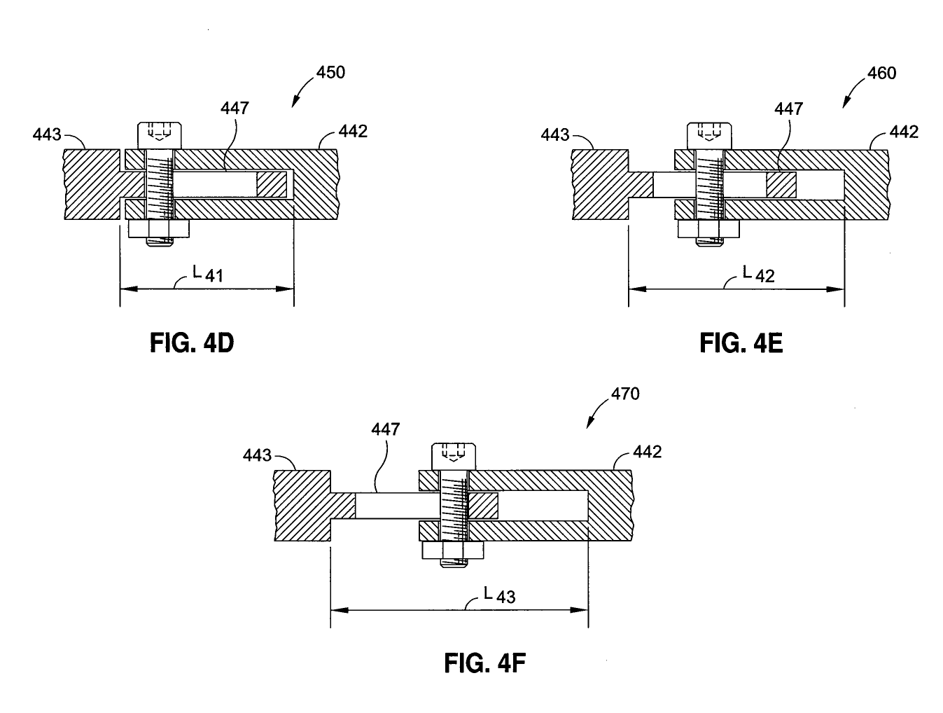

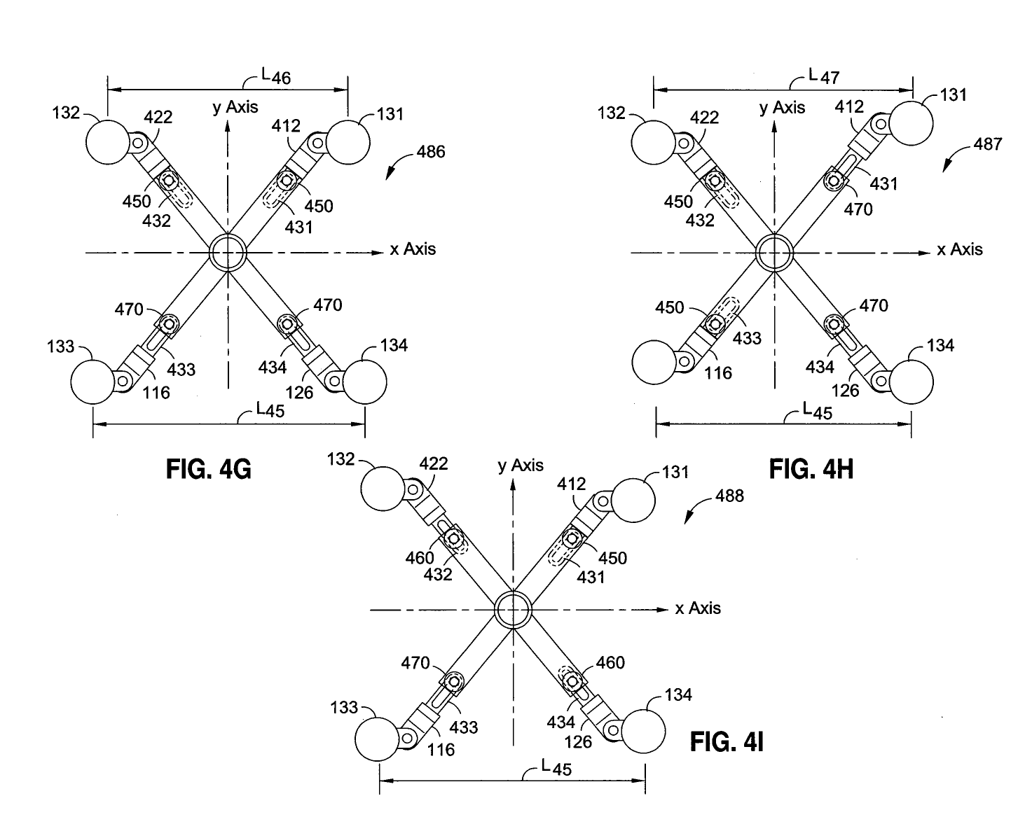

The discussion now turns to arm length adjusting feature of the Real-X cross connector. FIGS. 4A-4C show various views of a Real-X cross connector (RXCC) 400 with adjustable arms 410 and 420 according to an embodiment of the present invention. The RXCC 400 may be similar to the RXCC 100 in several aspects.

For example, the RXCC 400 may include a first elongated member (first arm) 410, a second elongated member (second arm) 420, the fulcrum member 130, and four connecting devices 131, 132, 133, and 134. The four connecting devices 131, 132, 133, and 134 may be implemented by the anchoring device 240 as shown in FIG. 2B, the articulated rod 340 as shown in FIG. 3B, or any other connecting devices, as long as they may connect the RXCC 400, directly or indirectly, to a set of readily anchored pedicle screws.

For another example, the first and second elongated members 410 and 420 may have first ends 412 and 422, second ends 416 and 426, and pivot segments 414 and 424. For another example, the fulcrum member 130 may engage and pivot the pivot segments 414 and 424, such that the first and second elongated members 410 and 420 may have a relative pivotal movement about the fulcrum member 130.

For yet another example, RXCC 400 may form an X-shape protection bridge, which may have similar structural and functional features as the X-shape protection bridge formed by the RXCC 100.

Despite these similarities, the RXCC 400 may be different from the RXCC 100 in at least one aspect. For example, the RXCC 400 may incorporate four arm length adjusting devices (ALADs) 431, 432, 433, and 434 to allow the first and second elongated members 410 and 420 to extend and/or retract their respective length. According to an embodiment of the present invention, the four ALADs 431, 432, 433, and 434 may share the structural and functional features of an ALAD 440 as shown in FIG. 4B-4C.

Generally, the ALAD 440 may include a locking screw 441, a nut member 448, a female member 442, and a male member 443. The female member 442 may be a receiving structure with a hollow core. As such, the female member 442 may include a top plate 444, a bottom plate 445 and a side wall 446. The side wall 446 may connect the top and bottom plates 444 and 445, which may define an opening and a space for receiving the male member 443. The male member 443 may have an insertion member 447 for inserting into the space of the female member 442.

In one embodiment, the female member 442 may be coupled to an end of the RXCC 400, which may be one of the first or second end 112, 122, 116, or 126, while the male member 443 may be coupled to the pivot segment 414 or 424. In another embodiment, the male member 443 may be coupled to an end of the RXCC 400, which may be one of the first or second ends 112, 122, 116, or 126, while the female member 442 may be coupled to the pivot segment 414 or 424.

Generally, the insertion member 447 may slide into or outside of the space of the female member 442 before the locking mechanism is triggered. In one embodiment, the insertion member 447 and the space may each have a length L₄₀, which may range, for example, from 2 mm to about 20 mm. As such, the ALAD 440 may have a retracted length which may range, for example, from about 2 mm to about 20 mm, as well as an extended length which may range, for example, from about 4 mm to about 40 mm.

After the female member 442 and the male member 443 are properly adjusted to achieve a desirable arm length, the locking mechanism may be triggered. Generally, the locking mechanism may be actuated by a coupling between the locking screw 441 and the nut member 448 or by any other methods that may affix the insertion member 447 within the space of the female member 442. As shown in FIG. 4C, the top and bottom plates 444 and 445 of the female member 442 may each have a penetration port for receiving the locking screw 441, and the insertion member 447 may have a narrow slit 449 for allowing the passage of the locking screw 441. In one embodiment, the locking screw 441 may pass through the opening of the top plate 444, then the narrow slit 449, and then the opening of the bottom plate 445.

After the locking screw 441 successfully penetrating the top plate 444, the insertion member 447 and the bottom plate 445, the nut member 448 may be coupled to the locking screw 441. Accordingly, a bolt of the locking screw 441 and the nut member 448 may apply a pair of compression forces against the top and bottom plates 444 and 445 respectively. The top and bottom plates 444 and 445 may then convert the pair of compression forces to a pair of frictional forces against the surfaces of the insertion member 447. As the pair of frictional forces increase, the insertion member 447 may become less free to slide along the space of the female member 442, and eventually, the insertion member 447 may be locked at a particular position.

FIGS. 4D-4F show the cross-sectional side views of several configurations of the ALAD 440 according to various embodiments of the present invention. As shown in FIG. 4D, the ALAD 440 may have a full retraction configuration, in which the insertion member 447 may be substantially inside of the space of the female member 442. As such, the ALAD 440 may have a fully retracted length L₄₁, which may be substantially the same as the length of the insertion member L₄₀. As shown in FIG. 4E, the ALAD 440 may have a partial extension configuration, in which the insertion member 447 may be partially inside of the space of the female member 442. As such, the ALAD 440 may have a partial extended length L₄₂, which may be greater than the fully retracted length L₄₁. As shown in FIG. 4F, the ALAD 440 may have a full extension configuration, in which the insertion member 447 may be substantially outside of the space of the female member 442. As such, the ALAD 440 may have a fully extended length L₄₃, which may be greater than the partial extended length L₄₂.

The aforementioned adjustment procedures and ALAD configurations may be applied to each of the ALADs 431, 432, 433, and 434. Advantageously, the RXCC 400 may have a dynamic range of arm length configurations for fitting patients with various spinal bone structures. FIGS. 4G-4I may help illustrate the benefit of the dynamic arm length configurations of the RXCC 400. For example, as shown in FIG. 4G, the RXCC 400 may have a symmetric-Y configuration 486 according to an embodiment of the present invention. With the symmetric-Y configuration 486, the RXCC 400 may be fitted to a patient with spinal bone structure that is symmetric along the Y-axis but asymmetric along the X-axis. More specifically, the first ALAD 431 may have the same arm length configuration 450 as the second ALAD 432 and the third ALAD 433 may have the same arm length configuration 470 as the fourth ALAD 434, while the first ALAD 431 may have a different arm length configuration as the third ALAD 433.

For another example, as shown in FIG. 4H, the RXCC 400 may have a symmetric-X configuration 487 according to an embodiment of the present invention. With the symmetric-X configuration 487, the RXCC 400 may be fitted to a patient with spinal bone structure that is symmetric along the X-axis but asymmetric along the Y-axis. More specifically, the first ALAD 431 may have the same arm length configuration 450 as the third ALAD 433 and the second ALAD 432 may have the same arm length configuration 470 as the fourth ALAD 434, while the first ALAD 431 may have a different arm length configuration as the second ALAD 432.

For yet another example, as shown in FIG. 4I, the RXCC 400 may have a fully asymmetric configuration 488 according to an embodiment of the present invention. With the fully asymmetric configuration 488, the RXCC 400 may be fitted to a patient with spinal bone structure that is asymmetric along the Y-axis and along the X-axis. More specifically, the first ALAD 431 may have a different arm length configuration from the second ALAD 432, which may have a different arm length configuration from the fourth ALAD 434.

It is understood that the X-axis and the Y-axis are relative terms and they should not be construed to represent any absolute orientation. For example, the Y-axis may be parallel to an approximate orientation of a patient's spine column. For another example, the X-axis may be parallel to the approximate orientation of the patient's spine column.

The discussion now turns to the structural and functional features of the fulcrum member 130. Generally, the fulcrum member 130 may be coupled to the pivot segments 114 and 124. As such, the fulcrum member 130 may perform as a pivot device for facilitating the pivotal movement between the first and second elongated members 110 (or 410) and 120 (or 420) as shown previously.

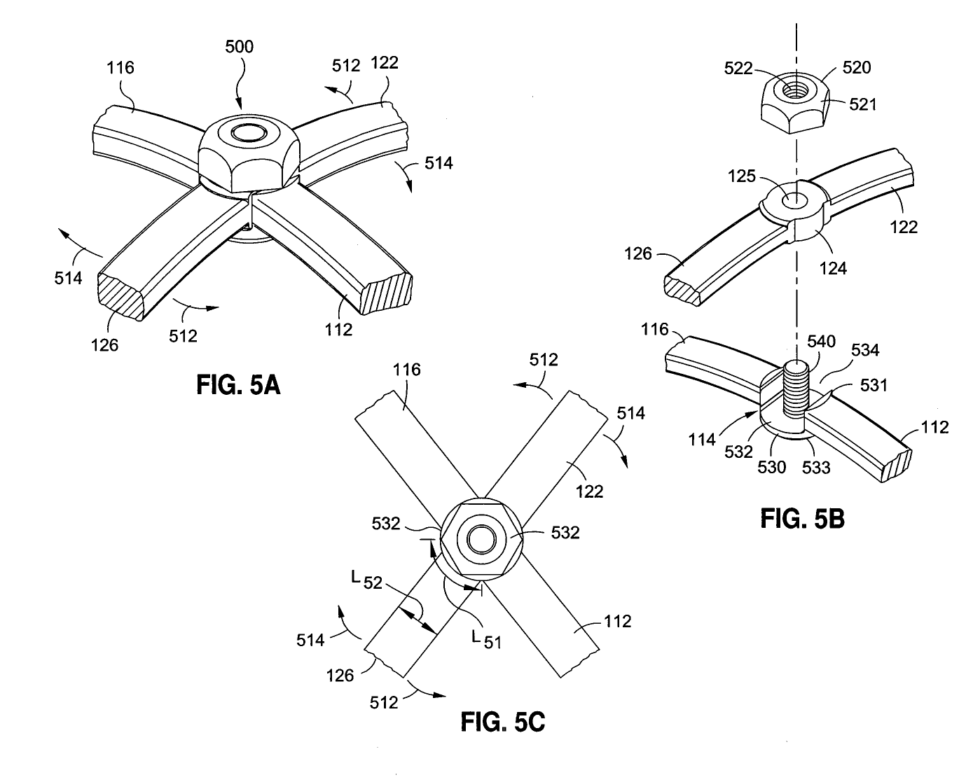

FIGS. 5A-5C show a perspective view, an exploded view, and a top view of a fulcrum member 500, which may be used to realize the fulcrum member 130 according to an embodiment of the present invention. Generally, the fulcrum member 500 may include a cover member 520, a base member 530, and a pivot pole member 540. The cover member 520 may have a top section 522 and an internal threaded section 521 formed along the inner surface cover member 520. The base member 530 may have a bottom section 533, a side wall 531 formed along the edge of the bottom section 533. Moreover, the base member 530 may be formed along the pivot segment 114 of the first elongated member 110, such that the side wall 531 may be attached, coupled, or connected to the first and second ends 112 and 116 of the first elongated member 110. Advantageously, the fulcrum member 500 may be partially integrated with the first elongated member 110 so that the number of assembly components, as well as the number of assembling steps, may be substantially reduced in forming the Real-X cross connector.

As shown in FIG. 5B, the side wall 531 may define a cylindrical space between the top section 521 and the bottom section 533, such that the pivot pin member 540 may be located along a central axis of the cylindrical space. Moreover, the side wall 531 may form a first receiving port 532 and a second receiving port 534 directly opposite to the first receiving port 532. Consequentially, the pivot segment 124 of the second elongated member 120 may be received within the cylindrical space and in between the first and second receiving ports 532 and 534.

As the pivot segment 124 of the second elongated member 120 descends into the receiving ports 532 and 534 of the base member 530, the pivot pin member 540 may penetrate a pivot hole 125 of the second elongated member 120, such that the pivot segment 114 of the first elongated member 110 may engage the pivot segment 124 of the second elongated member 120. When the pivot segment 124 is positioned substantially inside the cylindrical space, the cover member 520 may close the top space of the base member 530 by having the internal threaded section 522 to engage an external threaded section of the pivot pin member 540. Accordingly, the fulcrum member 500 may be formed, such that the second elongated member 120 and the first elongated member 110 may have the relative pivotal movement about the fulcrum member 500.

As shown in FIG. 5C, the second elongated member 120 may have a clockwise angular movement 514 and a counterclockwise angular movement 512 about the first and second openings 532 and 534. Generally, the first and second openings 532 and 534 may each have a width L₅₁ which may be wider than a width L₅₂ of the second elongated member 120. Accordingly, the range of clockwise and/or counterclockwise angular movements 512 and 514 of the second elongated member 120 may be controlled by a difference between the width L₅₁ and L₅₂.

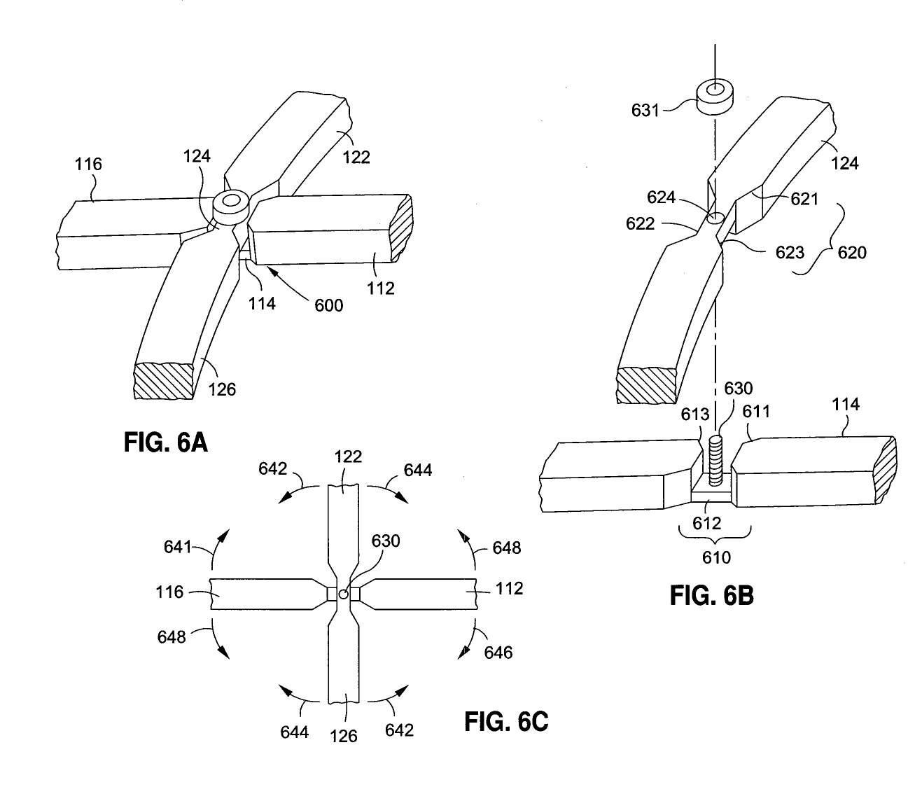

FIGS. 6A-6C show a perspective view, an exploded view, and a top view of an alternative fulcrum member 600, which may be used to realized the functions of the fulcrum member 130 according to an alternative embodiment of the present invention. Generally, the alternative fulcrum member 600 may include a first (bottom) joint member 610, a second (top) joint member 620, a pivot pin member 630 and a pivot cap member 631. As shown in FIGS. 6A and 6B, the first joint member 610 may be formed as part of the pivot segment 114, and the second joint member 620 may be formed as part of the pivot segment 124.

Accordingly, the first joint member 610 may be coupled to the first and second ends 112 and 116 of the first elongated member, and the second joint member 620 may be coupled to the first and second ends 122 and 126 of the second elongated member. Advantageously, the alternative fulcrum member 600 may be fully integrated with the first and second elongated members 110 and 120 so that the number of assembly components, as well as the number of assembling steps, may be substantially reduced.

More specifically, the first joint member 610 may have first and second buffer regions 611 and 613 and a middle bar 612, which may connect the first and second buffer regions 611 and 613. Similarly, the second member 620 may have first and second buffer regions 621 and 623 and a middle bar 622, which may connect the first and second buffer regions 621. In order to facilitate the proper coupling between the first and second joint members 610 and 620, the pivot pin member 630 may be formed on the middle bar 612, and a pivot hole 624 may be extended through the middle bar 622. Alternatively, the pivot pin member 630 may be formed on the middle bar 622, and a pivot hole (not shown) may be defined and extended through the middle bar 612 according to another embodiment of the present invention.

The second joint member 620 may engage the first joint member 610 by allowing the pivot hole 624 to slide down the pivot pin member 630. Because both the middle bars 612 and 622 may have a combined thickness that may be less than or equal to the thickness of the first elongated member 610 or the second elongated member 620, the middle bars 612 and 622 may be free from contacting each other. Additionally, an optional spacer (not shown) may be inserted between the middle bars 612 and 622 to provide additional stability between the first and second joint members 610 and 620. After the first and second joint members 610 and 620 are properly coupled, the pivot cap 631 may be secured to the pivot pin 630 for locking the first and second joint members 610 and 620 together.

As shown in FIG. 6C, the first and second ends 112 and 116 of the first elongated member 610 may have clockwise and counterclockwise angular movements 646 and 648 about the pivot pin member 630. Similarly, the first and second ends 122 and 126 of the second elongated member 620 may have clockwise and counterclockwise angular movements 644 and 642 about the pivot pin member 630. Because the first and second buffer regions 611,621, 613, and 623 may be slightly sloped, the impact between the first and second elongated members 610 and 620 may be substantially minimized.

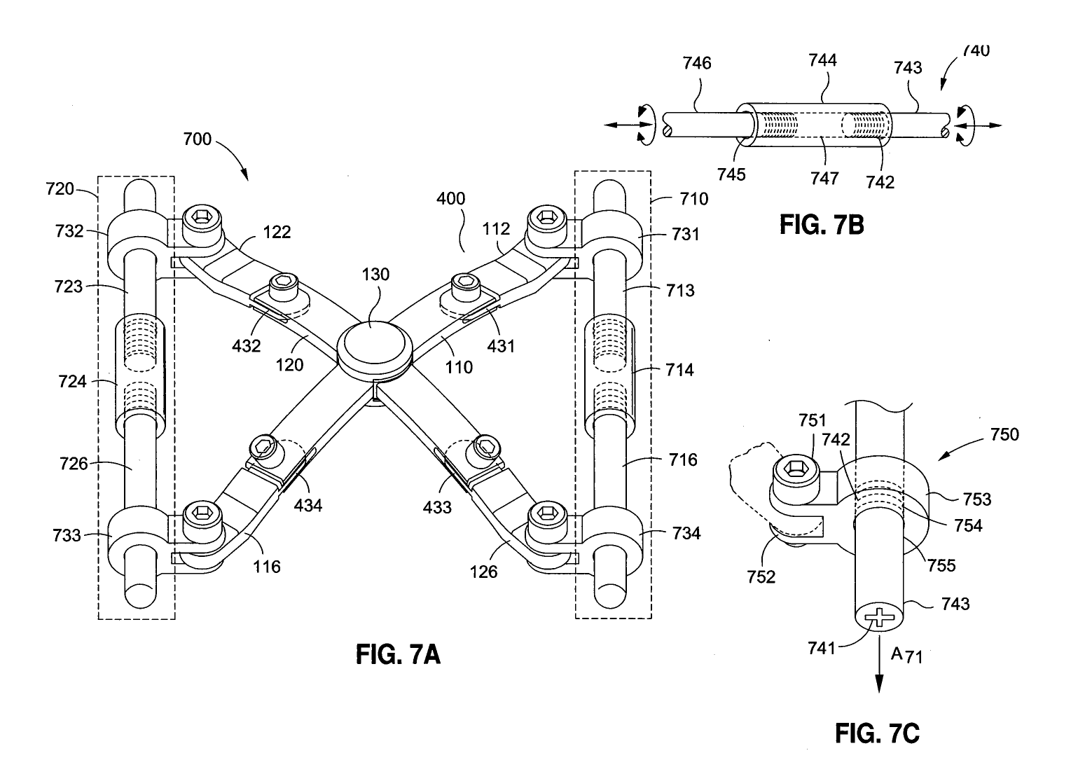

FIGS. 7A-7C show various views of a Real-X cross connector (RXCC) 700 with first and second adjustable rod assemblies (ARAs) 710 and 720 as the connecting devices according to an embodiment of the present invention. Generally, the RXCC 700 may incorporate several structural and functional features of the RXCC 400. For example, the RXCC 700 may incorporate the X-shape protection bridge and the benefits thereof. For another example, the RXCC 700 may incorporate the arm length adjustable devices (ALADs) 431, 432, 433, and 433, and the benefits thereof Like the RXCC 400, the RXCC 700 may have a dynamic range of arm length configurations for patients with various spinal bone structures.

Despite these similarities, the RXCC 700 may be different from the RXCC 400 in at least one aspect. For example, the RXCC 700 adopted two ARAs 710 and 720 as the connecting devices according to an embodiment of the present invention. From a design standpoint, the ARAs 710 and 720 may provide an integrated solution for conventional cross connectors.

Mainly, the ARAs 710 and 720 may incorporate the structural and functional features of the pair of stabilizing rods 162 and 164 as shown in FIG. 1E as well as the structural and functional features of the several connecting devices discussed so far. As such, the RXCC 700 may be pre-assembled and pre-adjusted according to a surgeon's assessment of a patient's spinal bone structure before the actual spinal fixation surgery is being performed. Advantageously, the ARAs 710 and 720 may improve conventional spinal fixation surgery by reducing the number of surgical steps, the time spent on performing the surgery, and the surgical risk associates with the lengthy surgical procedures.

As shown in FIG. 7A, the first ARA 710 may include first and second articulated ring members 731 and 734, first and second rod segments 713 and 716, and a rod adjustment device 714. Particularly, the first articulated ring member 731 may engage the first rod segment 713, the second articulated ring member 734 may engage the second rod segment 716, and the rod adjustment device 714 may be engaged to both the first and second rod segments 713 and 716. Moreover, the first articulated ring member 731 may be coupled to the first end 112 of the first elongated member 110, and the second articulated ring member 734 may be coupled to the second end 126 of the second elongated member 120.

Similar to the first ARA 710, the second ARA 720 may include first and second articulated ring members 732 and 733, first and second rod segments 723 and 726, and a rod adjustment device 724. Particularly, the first articulated ring member 732 may engage the first rod segment 723, the second articulated ring member 733 may engage the second rod segment 726, and the rod adjustment device 724 may be engaged to both the first and second rod segments 723 and 726. Moreover, the first articulated ring member 732 may be coupled to the first end 122 of the first elongated member 120, and the second articulated ring member 733 may be coupled to the second end 116 of the second elongated member 110.

According to an embodiment, the functions of the rod adjustment devices 714 and 724 may be realized by a rod adjustment assembly 740 as shown in FIG. 7B. Generally, the rod adjustment assembly 740 may include a sleeve member 744, a first insertion member 743, and a second insertion member 746. Particularly, the first insertion member 743 may be coupled to the first rod segment 713 or the first rod segment 723, and the second insertion member 746 may be coupled to the second rod segment 716 or the second rod segment 726.

More particularly, the first and second insertion member 743 and 746 may have external threaded surfaces 742 and 745 respectively, and the sleeve member 744 may have an internal threaded surface 747. When the external threaded surfaces 742 and 745 engage the internal threaded surface 747, the first and second insertion members 743 and 746 may be screwed into or out of the sleeve member 744. Accordingly, the rod adjustment assembly 740 may have an adjustable length depending on the relative positions of the first and second rod segments 743 and 746 with respect to the sleeve member 744.

In one embodiment, the function of the articulated ring members 731, 732, 733, and 734 may be realized by an articulated ring assembly 750 as shown in FIG. 7C. Generally, the articulated ring assembly 750 may have a locking screw 751, a joint member 752, and a ring member 753. Particularly, the joint member 752 may cooperate with the locking screw 751 for engaging and securing one of the first or second end 112, 122, 116, or 126. Depending on the design goal, the joint member 752 may be permanently or temporarily coupled to the ring member 753.

The ring member 753 may have a receiving port 755 for receiving a rod segment 743, which may be one of the first rod segment 713 of the first ARA 710, the second rod segment 716 of the first ARA 710, the first rod segment 723 of the second ARA 720, or the second rod segment 726 of the second ARA 720. Moreover, the ring member 753 may have one or more locking mechanism for preventing the rod segment 743 from sliding pass the receiving port 755 while allowing the rod segment 743 to have a free rotational movement about its central axis A₇₁.

To implement the locking mechanism, the ring member 753 may include one or more protrusion ring(s) 754 disposed along the inner surface of the receiving port 755 according to an embodiment of the present invention. As shown in FIG. 7C, the rod segment 741 may have one or more corresponding intrusion ring(s) 741 for engaging the one or more protrusion ring(s) 754 of the ring member 753. Advantageously, the rod segment 743 may be rotated about the central axis A₇₁ while being secured by the ring member 753.

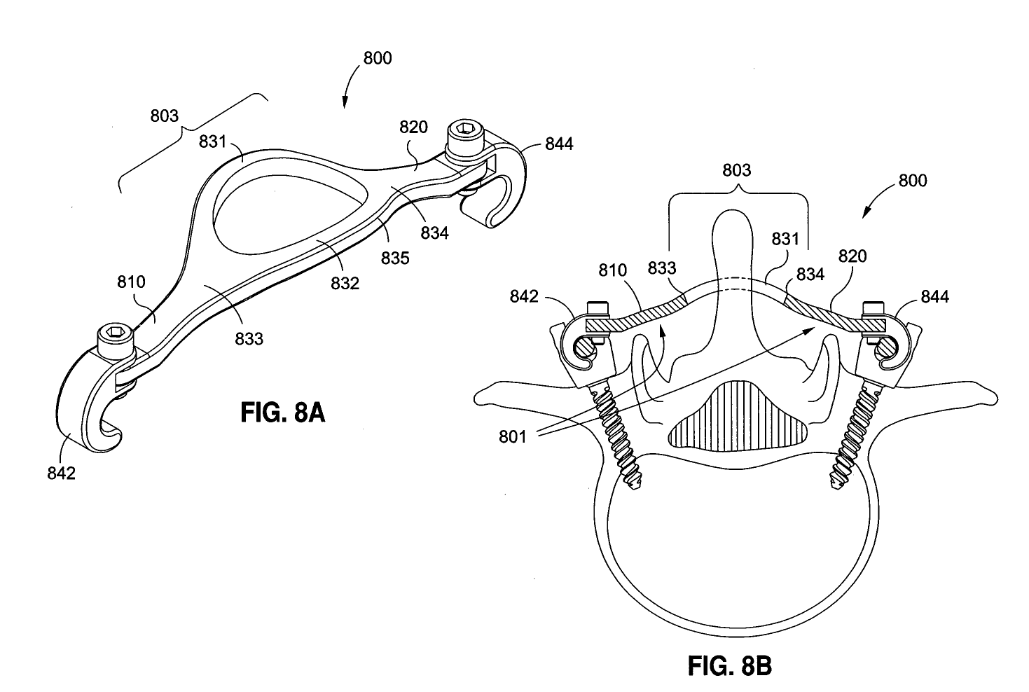

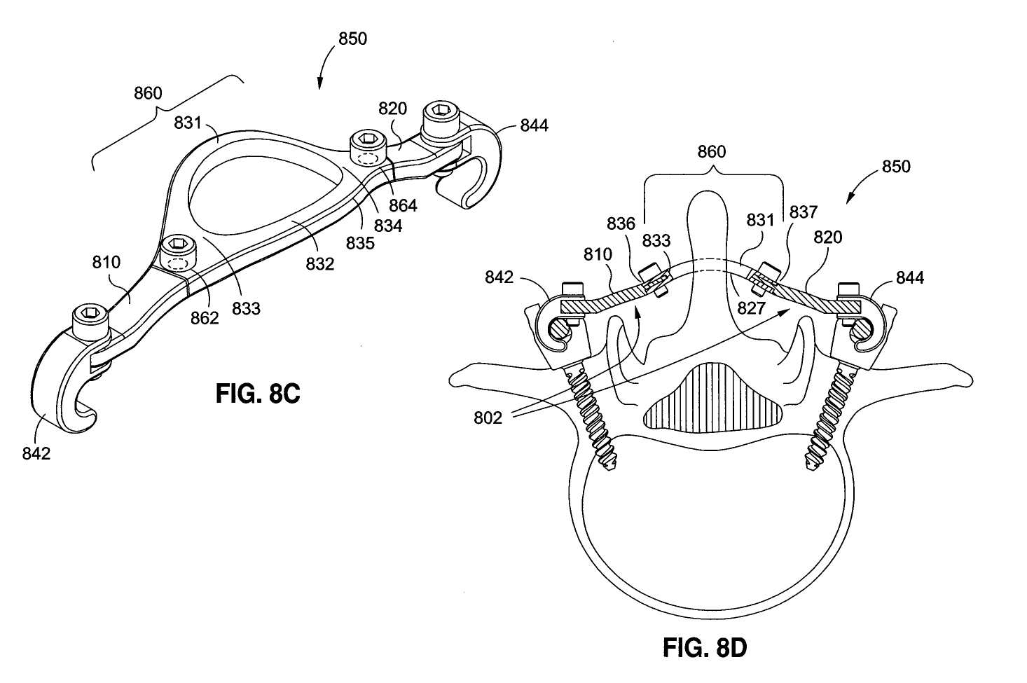

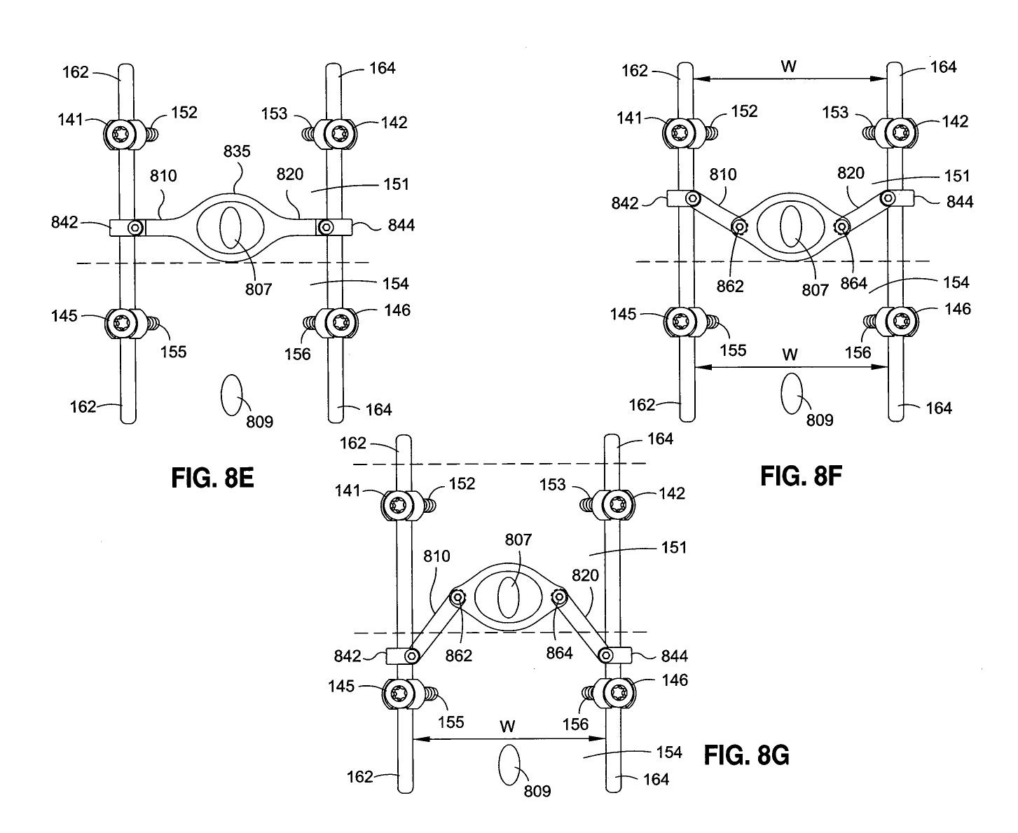

The discussion now turns to a Real-O cross connector (ROCC), which may be used as an alternative device of the Real-X cross connector as discussed previously. FIGS. 8A-8B show a perspective view and a cross sectional side view of a ROCC 800 according to an embodiment of the present invention. Generally, the ROCC 800 may include a center member 803, a first arm 810 and a second arm 820, and first and second anchoring devices 842 and 844. Particularly, the first and second anchoring devices 842 and 844 may be coupled to the first and second arms 810 and 820 respectively. The first and second anchoring devices 842 and 844 may be used for anchoring the ROCC 800 to two stabilizing rods, which may be anchored to several spinal bone segments by several pedicle screws. Accordingly, the structural and functional features of the first and second anchoring devices 842 and 844 may be realized by the anchoring device 240 of FIG. 2B.

In one embodiment, the first and second arm 810 and 820 may be connected to the center member 803 to form an arch bridge 801 as shown in FIG. 8B. The center member 803 may include first and second ends 833 and 834, and first and second bracket 831 and 832, which may join each other at the first and second ends 833 and 834. Together, the first and second brackets 831 and 832 may form a protection ring 835 at the center of the ROCC 800.

The arch bridge 801 may define a space underneath the center member 803, and the protection ring 835 may create an opening at the center of the ROCC 800. Hence, the ROCC 800 may be place direct above a spinal bone segment and may avoid contacting the spinal bone segment's superior articular process, Mamillary process, accessory process, and inferior articular process. Furthermore, the protection ring 835 may help protect and preserve the spinous process by laterally surrounding a base of the spinous process, such that the spinous process of the spinal bone segment may protrude from the protection ring 835. Advantageously, the ROCC 800 may be placed directly across the spinal bone segment without removing the spinous process thereof, and thus, the ROCC 800 may also help prevent symptoms of pseudoarthritis.

Referring to FIG. 8E, the ROCC 800 may be anchored to and positioned in between the first and second stabilizing rods 162 and 164 according to an embodiment of the present invention. Generally, the first stabilizing rod 162 may be anchored to the left pedicles 152 and 155 via the pedicle screws 141 and 145, while the second stabilizing rod 164 may be anchored to the right pedicles 153 and 156 via the pedicle screws. As such, the first and second stabilizing rods 162 and 164 may provide a vertical stabilization for the spinal bone segments 151 and 154.

In order to provide a horizontal stabilization, the ROCC 800 may be anchored to the first stabilizing rod 162 by using the first anchoring device 842 and to the second stabilizing rod 164 by using the second anchoring device 844. Because of the opening defined by the protection ring 835 and the space underneath the arched bridge 801, the ROCC 800 may be conveniently placed above and across the spinal bone segment 151 without removing the spinous process 807 thereof. Advantageously, the ROCC 800 may improve the conventional spinal fixation surgery by making it safer and less intrusive to the patient's body. The above procedure may be repeated for other spinal bone segments. For example, another ROCC 800 may be placed above and across the spinal bone segment 154, such that the protection ring 835 may be placed around the base section of the spinous process 809.

FIG. 8C-8D show a perspective view and a cross-sectional of an alternative ROCC 850 according to another embodiment of the present invention. Generally, the ROCC 850 may share several structural and functional features with the ROCC 800. For example, the ROCC 850 may have the first and second arms 810 and 820, the first and second anchoring devices 842 and 844, and a center member 860, which may be connected between the first and second arms 810 and 820. For another example, the center member 860 of the ROCC 850 may include the first and second brackets 831 and 832, which may be joined at the first and second ends 833 and 834 respectively to form the protection ring 835. Moreover, the ROCC 850 may form an arched bridge 802, which may have similar structure and provide similar functionalities as the arched bridge 801.

Despite these similarities, the ROCC 850 may be different from the ROCC 800 in at least one aspect. For example, the center member 860 of the ROCC 850 may include a first joint member 862 for engaging the first arm 810 and a second joint member 864 for engaging the second arm 820. Generally, the first and second joint member 862 and 864 may function as two pivoting devices for the protection ring 835.

More specifically, the first and second joint member 862 and 864 may include certain joint mechanism to allow each of the first and second arms 810 and 820 to have a range of angular movement about the first and second ends 833 and 834 so that the ROCC 850 may be adjusted to adapt to various spinal bone structures. Meanwhile, the first and second joint member 862 and 864 may include certain locking mechanism to lock each of the first and second arms 810 and 820 once the ROCC 850 is properly adjusted. In one embodiment, for example, the functional features of the joint members 862 and 863 may be implemented by the joint member 242 as shown and discussed in FIG. 2B.

Referring to FIG. 8F-8G, the ROCC 850 may be anchored to and positioned in between the first and second stabilizing rods 162 and 164 according to an embodiment of the present invention. Generally, the first stabilizing rod 162 may be anchored to the left pedicles 152 and 155 via the pedicle screws 141 and 145, while the second stabilizing rod 164 may be anchored to the right pedicles 153 and 156 via the pedicle screws 142 and 146. As such, the first and second stabilizing rods 162 and 164 may provide the vertical stabilization for the spinal bone segments 151 and 154, and the ROCC 850 may provide the horizontal stabilization for the first and second stabilizing rods 162 and 164.

In addition to the advantages of the ROCC 800, the ROCC 850 may include other advantages. For example, the joint members 862 and 864 may provide the ROCC 850 with more adjustability in terms of selecting the pair of anchoring points. As shown in FIG. 8F, each of the spinal bone segments 151 and 154 may have a bone width W, which may be shorter than the combined length of the first and second arms 810 and 820. Because the joint members 862 and 864 allow the first and second arms 810 and 820 to fold up or down from the center member 860, the anchoring devices 842 and 844 may established various anchor points along the first and second stabilizing rods 162 and 164.

In order to adapt to the narrow spinal bone segments 151 and 154, the first and second arms 810 and 820 may be folded upward to reach a pair of higher anchored points, so as to reduce the distance between the protection ring 835 and the first and second stabilizing rods 162 and 164. This adjustment process may be repeated for adapting the ROCC 850 to spinal bone segments with a range of spinal bone widths. Advantageously, the ROCC 850 may be installed to patients with spinal bone segments of various widths.

Furthermore, the adjustability provided by the first and second joint members 862 and 864 may allow the ROCC 850 to adapt to asymmetric spinal bone segments. As shown in FIG. 8G, the spinous process 807 of the spinal bone segment 151 may be closer to the left pedicle 152 than to the right pedicle 153. In order to adapt to the asymmetry of the spinal bone segment 152, the first arm 810 may be folded with a larger downward angle than the second arm 820. Accordingly, the distance between the protection ring and the first stabilizing rod 162 may be less than the distance between the protection ring and the second stabilizing rod 164. This adjustment process may be repeated for adapting the ROCC 850 to spinal bone segments with various degrees of asymmetry. Advantageously, the ROCC 850 may be applied to fit patients with asymmetric spinal bone segments.

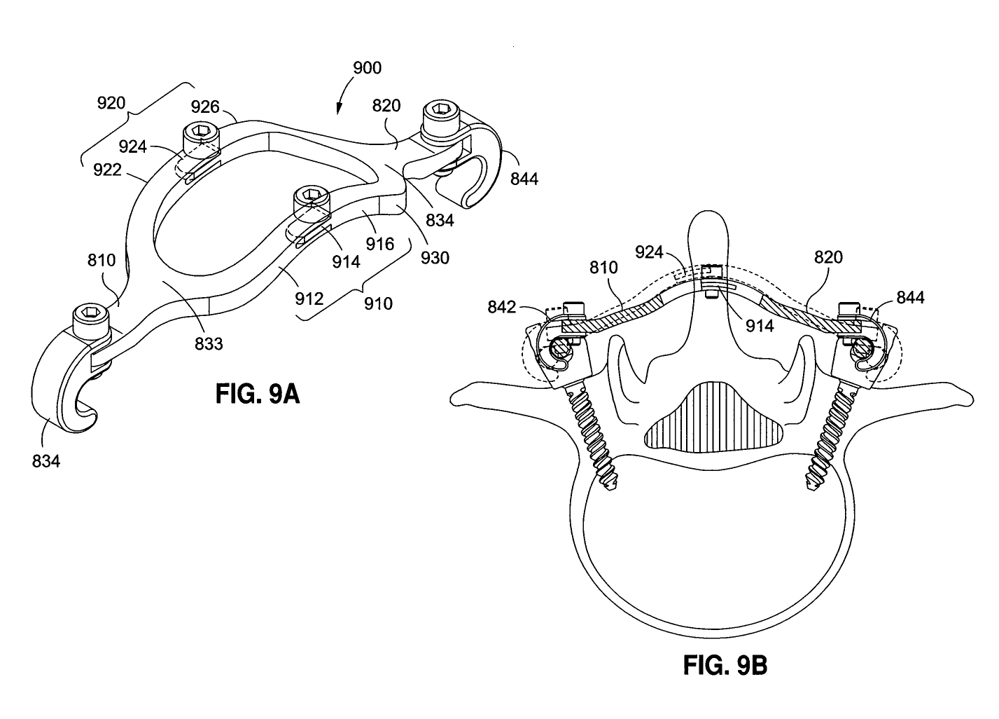

FIGS. 9A-9B show various views of a Real-O cross connector (ROCC) 900 with an adjustable ring according to an embodiment of the present invention. Generally, the ROCC 900 may incorporate the structural and functional features of the ROCC 800 and/or the ROCC 850. Additionally, the ROCC 900 may include an adjustable center member 930 in replacing the center member 803 and/or 860. The adjustable center member 930 may include a first adjustable bracket 910 and a second adjustable bracket 920. More particularly, the first and second adjustable brackets 910 and 920 may have first segments 912 and 922, second segments 916 and 926, and length adjustable devices 914 and 924.

The length adjustable device 914 may engage the first and second segments 912 and 916 of the first adjustable bracket 910, and the length adjustable device 914 may change the relative position between the first and second segments 912 and 916. Accordingly, the length adjustable device 914 may change the length of the first adjustable bracket 910. Similarly, the length adjustable device 924 may engage the first and second segments 922 and 926 of the first adjustable bracket 920, and the length adjustable device 924 may change the relative position between the first and second segments 922 and 926. Accordingly, the length adjustable device 924 may change the length of the first adjustable bracket 920.

The functional features of the length adjustable devices 914 and 924 may be realized by any compatible mechanical components. In one embodiment, for example, the length adjustable devices 914 and 924 may each be implemented by the arm length adjustable device 440 as described and discussed in FIGS. 4B-4F.

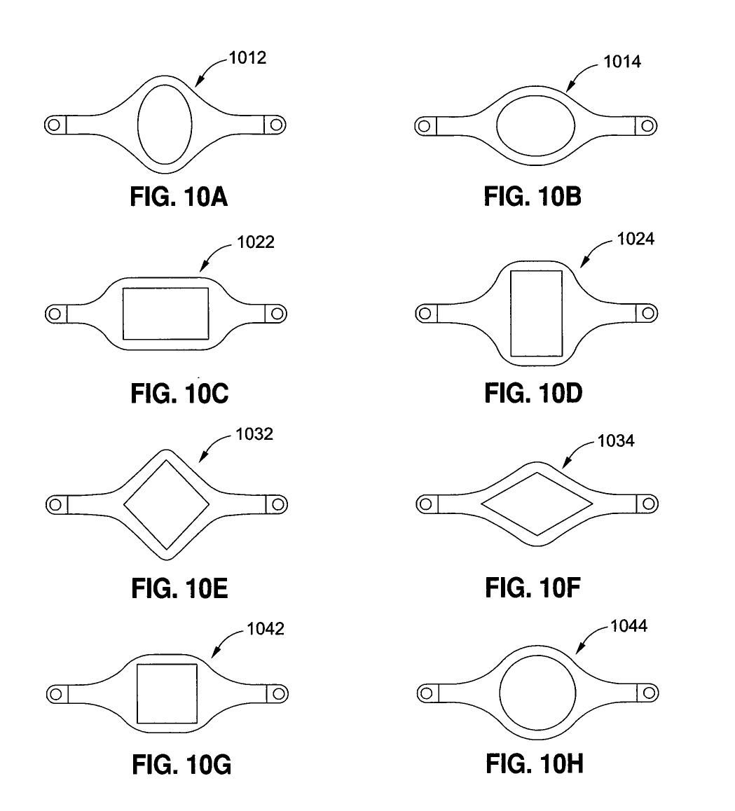

The discussion now turns to the various shapes of the protection rings of the Real-O cross connectors according to various embodiments of the present invention. As shown in FIG. 10A, the protection ring 1012 may, for example, have a shape of a vertical oval. As shown in FIG. 10B, the protection ring 1014 may, for example, have a shape of a horizontal vertical oval. As shown in FIG. 10C, the protection ring 1022 may, for example, have a shape of a horizontal rectangle. As shown in FIG. 10D, the protection ring 1024 may, for example, have a shape of a vertical rectangle. As shown in FIG. 10E, the protection ring 1032 may, for example, have a shape of a vertical rhombus. As shown in FIG. 10F, the protection ring 1034 may, for example, have a shape of a horizontal rhombus. As shown in FIG. 10G, the protection ring 1042 may, for example, have a shape of a square. As shown in FIG. 10H, the protection ring 1044 may, for example, have a shape of a circle. The aforementioned shapes of the protection rings are only for illustrative purpose since the protection ring may have other shapes that may be adaptive to various contour of the base section of the spinous process.

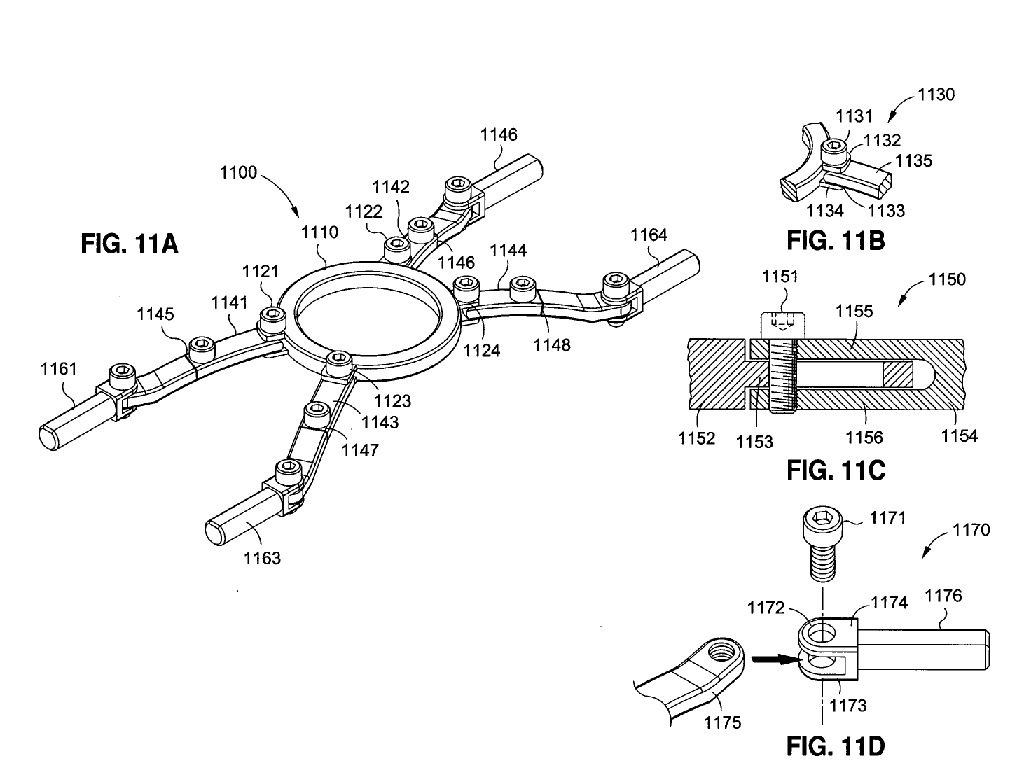

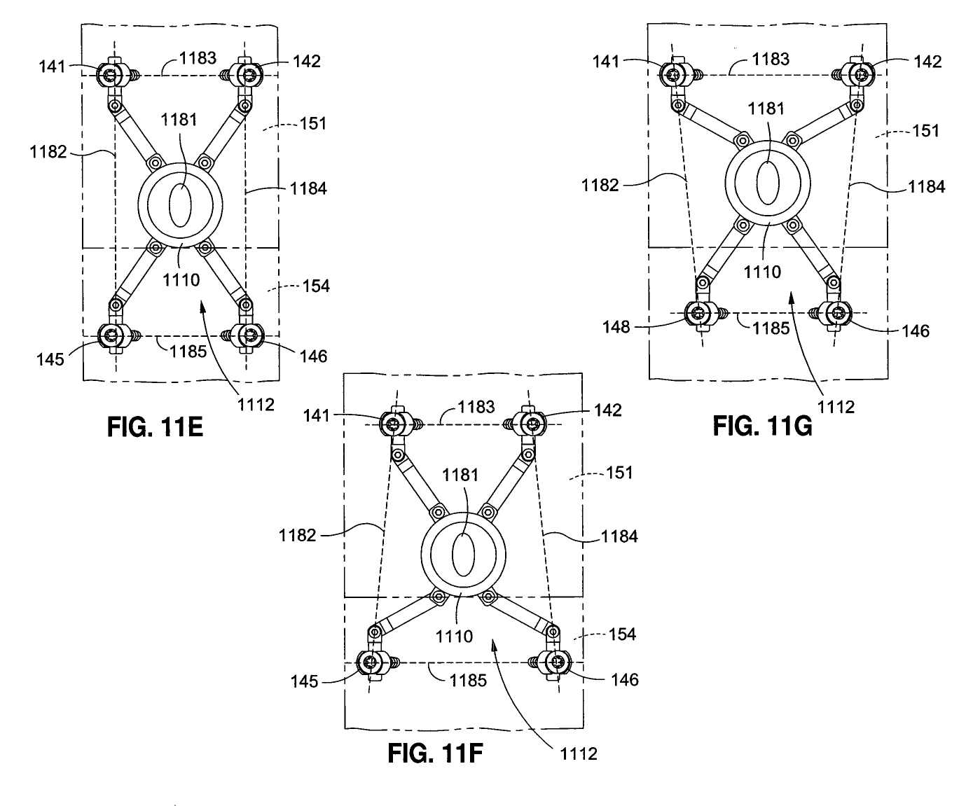

The discussion now turns to a Real-XO cross connector (RXOCC), which may be used as an alternative device of the Real-X cross connector (RXCC) and the Real-O cross connector (ROCC). FIGS. 11A-11D show various views of an RXOCC 1100 according to an alternative embodiment of, the present invention. Generally, the RXOCC 1100 may incorporate several structural and functional features of the Real-X cross connectors (RXCC) and the Real-O cross connectors (ROCC) as discussed previously. For example, the RXOCC 1100 may include a protection ring 1110, four joint members 1121, 1122, 1123, and 1124, four elongated members 1141, 1142, 1143, and 1144, four arm length adjustable devices (ALADs) 1145, 1146, 1147, and 1148, and four connecting devices 1161, 1162, 1163, and 1164.

In one embodiment, the joint members 1121, 1122, 1123, and 1124 may secure the elongated members 1141, 1142, 1143, and 1144 to the protection ring 1110. In another embodiment, the ALADs 1145, 1146, 1147, and 1148 may be adjustable so that the elongated members 1141, 1142, 1143, and 1144 may each have an adjustable length. In yet another embodiment, the connecting devices 1161, 1162, 1163, and 1164 may connect the RXOCC to one or more spinal bone segments via several pedicle screws and/or a pair of elongated stabilizers. Although the connecting devices 1161, 1162, 1163, and 1164 are implemented by the articulated rod 1170 as shown in FIG. 11A, they may be implemented by other devices, such as the anchoring device 240 as shown in FIG. 2B.

Specifically, the elongated members 1141, 1142, 1143, and 1144 may be distributed along the edge of the protection ring 1110. When the joint members 1121, 1122, 1123, and 1124 are unlocked, the elongated members 1141, 1142, 1143, and 1144 may be free to be angularly displaced about the respective joint members. Alternatively, the elongated members 1141, 1142, 1143, and 1144 may be free to move along the edge of the protection ring 1110 when the respective joint members 1121, 1122, 1123, and 1124 are unlocked. When the joint members 1121, 1122, 1123, and 1124 are locked, the elongated members 1141, 1142, 1143, and 1144 may each be affixed to a particular position in relative to the protection ring 1110.

At the locking mode, the RXOCC 1100 may form a hybrid X-shaped protection bridge, which may arch over a space directly underneath the protection ring 1110 while allowing the space to extend through an opening defined by the protection ring 1110. Advantageously, the hybrid X-shaped protection bridge may inherit the benefits of the Real-X cross connector (RXCC) and the Real-O cross connector (ROCC).

As shown in FIG. 11B, the four joint members 1121, 1122, 1123, and 1124 may each be implemented by a lockable joint 1130 according to an embodiment of the present invention. The lockable joint 1130 may include a locking screw 1131, a first plate 1132, a second plate 1133, and a side body 1134. The side body 1134 may be coupled to the edge of the protection ring 1110, such that the lockable joint 1130 may receive an end member 1135 along an outer circumferential surface (the edge) of the protection ring 1110. As discussed herein, the end member 1135 may be one of the first, second, third, or fourth elongated member 1141, 1142, 1143, or 1144. Moreover, the first and second plates 1132 and 1133 may be separated by a space for receiving the end member 1135, and they may each have an opening for receiving the locking screw 1131.

Before the locking screw 1131 substantially engages the second plate 1133, the end member 1135 may be freely rotated about the locking joint member 1130. Correspondingly, the first, second, third, and fourth elongated members 1141, 1142, 1143, and 1144 may be adjusted to different angular positions with respect to the protection ring 1110. Advantageously, the RXOCC 1100 may be adjustable to form X-shape protection bridges with various angular positions.

In order to lock the lockable joint 1130, the locking screw 1131 may be used for substantially engaging the second plate 1133. The locking screw 1131 may cooperate with the second plate 1133 to produce a pair of compression forces, which may be asserted against the end member 1135. As such, the frictional forces between the end member 1145 and the inner surfaces of the first and second plates 1132 and 1133 may be increased significantly. As a result, the end member 1135 may be locked in a particular position with respect to the lockable joint member 1130. Correspondingly, the first, second, third, and fourth elongated members 1141, 1142, 1143, and 1144 may each be locked at a particular angularly position with respect to the protection ring 1110.

FIG. 11C shows a cross-sectional side view of an ALAD 1150, which may realize the functional features of the first, second, third and fourth ALADs 1145, 1146, 1147, and 1148. In one embodiment, for example, the ALAD 1150 may include the same components as the ALAD 440 (see FIGS. 4B and 4C), and it may thus incorporate the functional features of the ALAD 440. Generally, the ALAD 1150 may include a locking screw 1151 a male member 1152, which may have an insertion member 1153, a female member 1154, which may have first and second plates 1155 and 1156 to define a space for receiving the insertion member 1153.

More specifically, the insertion member 1153 may be slid in and out of the space before the locking screw 1151 substantially engages the second plate 1156. As such, the distance between the male and female member 1152 and 1154 may be adjusted. However, when the locking screw 1151 substantially engages the second plate 1156, the insertion member 1153 may be locked within a particular position within the space defined within the female member 1154. Accordingly, the male and female members 1152 and 1154 may be substantially stabilized and they may thus form an adjusted distance between them.

FIG. 11D shows a cross-sectional side view of an articulated rod 1170, which may realize several functional features of the first, second, third, and fourth connecting devices 1161, 1162, 1163, and 1164 as discussed earlier. In one embodiment of the present invention, for example, the articulated rod 1170 may include the same components as the articulated rod 340 (see FIGS. 3B and 3C), and it may thus incorporate the functional features of the articulated rod 340. Generally, the articulated rod 1170 may include a lockable joint member 1174 and a rod member 1176, which may be connected to the lockable joint member 1174.

The lockable joint member 1174 may be similar to the lockable joint member 1130. As such, the lockable joint member 1174 may be used to secure an end member 1175, which may be one of the first, second, third, or fourth elongated member 1141, 1142, 1143, or 1144. Specifically, the locking joint member 1171 may include first and second plates 1172 and 1173, which may define a space for receiving the end member 1175, and a locking screw 1171 for locking the end member 1175 between the first and second plates 1172 and 1173. The rod member 1176 may share similar functionalities as a conventional stabilizing rod such that the rod member 1176 may be received and secured by a conventional pedicle screw, which may be anchored to a spinal bone segment.

Because the RXOCC 1100 may be fully adjustable before the several locking mechanisms are applied, the X-shape protection bridge 1112 may have several configurations for fitting patients with various spinal bone structures. In FIG. 11E, the spinal bone segments 151 and 154 may have a pair of parallel inter-segment lines and a pair of parallel intra-segment lines. The pair of inter-segment lines may include a first inter-segment line 1182 defined by the pedicle screws 141 and 145, and a second inter-segment line 1184 defined by the pedicle screws 142 and 146. Moreover, the pair of intra-segment lines may include a first intra-segment line 1181 defined by the pedicle screws 141 and 142, and a second intra-segment line 1185 defined by the pedicle screws 145 and 146. As such, the X-shape protection bridge may have a fully symmetrical configuration according to an embodiment of the present invention, and in which the protection ring 1110 may surround a base section of a spinous process 1181 of the spinal bone segment 151.

Referring to FIG. 11F, the spinal bone segments 151 and 154 may have a pair of diverging intra-segment lines 1182 and 1184 and a pair of parallel inter-segment lines 1183 and 1185. As such, the X-shape protection bridge may be adjusted to have a partial symmetrical configuration according to another embodiment of the present invention. Referring to FIG. 11G, the spinal bone segments 151 and 154 may have a pair of diverging intra-segment lines 1182 and 1184 and a pair of diverging inter-segment lines 1183 and 1185. As such, the X-shape protection bridge may be adjusted to have a fully asymmetrical configuration according to yet another embodiment of the present invention.

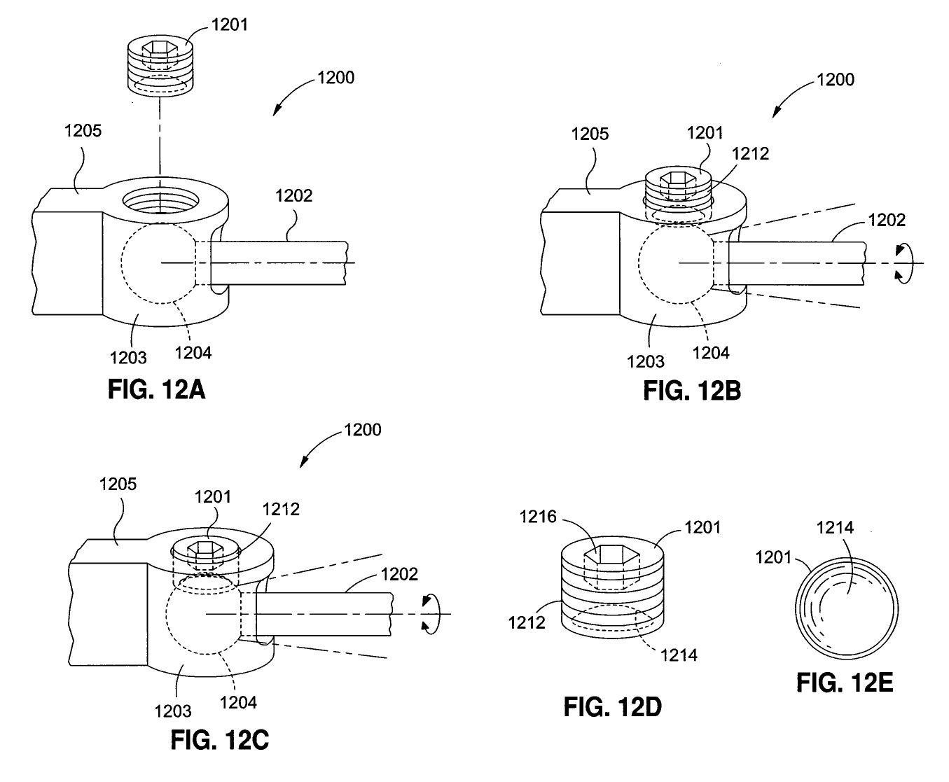

The discussion now turns to an alternative lockable joint member. Although the lockable joint member with the two-plate configuration has been discussed with respect to various embodiments of the present invention, an alternative lockable joint member with a multi-axial joint may be used for realizing several functional features of the lockable joint member. As shown in FIG. 12A, an alternative lockable joint member 1200 may generally include a locking screw 1201, a housing 1205, a socket 1203 located within the housing 1202, a bearing 1204, and a handle member 1202. More specifically, the housing may have a top surface and a side wall, such that a top receiving port may be formed on the top surface and a side receiving port may be formed on the side wall.

As shown in FIG. 12B, the socket 1203 may receive the bearing 1204, and it may have a socket surface for contacting the bearing 1204 and thereby allowing the bearing 1204 to rotate therein. The handle member 1202 may be coupled to the bearing 1204 and it may protrude from the side wall of the housing 1205 via the side receiving port. The handle member 1202 may have a range of multi-axle movement about a center of the bearing 1204 or about the side receiving port. Depending on the other functions of the lockable joint member 1200, the housing 1205 may be coupled to a rod member in one embodiment or a hook member in another embodiment. The handle member 1202 may be coupled to an end of an elongated member (arm), such that the housing 1205 may rotate about the end of the elongated member.

As shown in FIG. 12C, the locking screw 1201 may descend into the top opening of the housing 1205. When the external threaded section 1212 of the locking screw 1201 substantially engages the internal threaded section of the housing 1205, the inner concave surface 1214 may assert a compression force against the bearing 1204. Consequentially, the compression force may cooperate with the surface of the socket 1203 to lock the bearing 1204 at a particular position.

As shown in FIG. 12D, the locking screw 1201 may have a bearing socket 1216 for receiving a driving force. The driving force may cause the external threaded section 1212 of the locking screw 1201 to substantially engage the internal threaded section of the housing 1205. In FIG. 12E, which shows the bottom view of the locking screw 1201, the bottom concave surface 1214 may be used for engaging the bearing 1204 and thus locking the bearing 1204 in a particular position. In one embodiment, the bottom concave surface 1214 may be distributed with compressible rings. In another embodiment, the bottom concave surface 1214 may be distributed with small protrusions. In yet another embodiment, the inner concave surface 1214 may be a rough surface, which may cause a significant amount of friction upon contact.

Exemplary embodiments of the invention have been disclosed in an illustrative style. Accordingly, the terminology employed throughout should be read in a non-limiting manner. Although minor modifications to the teachings herein will occur to those well versed in the art, it shall be understood that what is intended to be circumscribed within the scope of the patent warranted hereon are all such embodiments that reasonably fall within the scope of the advancement to the art hereby contributed, and that scope shall not be restricted, except in light of the appended claims and their equivalents.

What is claimed is:

1. A cross connector for use in conjunction with four or more pedicle screws for stabilizing and protecting one or more fixation levels of spinal bone segments, the cross connector configured to be anchored to the spinal bone segments by the four or more pedicle screws, the cross connector comprising:
first and second elongated members each having first and second ends and a pivot segment positioned between the first and second ends;
a fulcrum member configured to engage the pivot segment of the first elongated member and the pivot segment of the second elongated member, thereby allowing a relative movement therebetween; and
a plurality of connecting devices, each configured to connect one of the first end or the second end of one of the first elongated stabilizer or the second elongated stabilizer to one of the four or more pedicle screws, such that the first and second elongated members are configured to form an X-shape bridge across the one or more fixation levels of spinal bone segments.

2. The cross connector of claim 1, wherein the X-shape bridge is configured to arch over the one or more fixation levels of spinal bone segments.

3. The cross connector of claim 2, wherein each of the first and second elongated members has:
an arched section along the pivot segment, and
first and second flat sections along the first and second ends.

4. The cross connector of claim 1, wherein the first and second elongated members each has an adjustable length.

5. The cross connector of claim 1, wherein the relative movement between the first and second elongated members is substantially restricted after the first and second ends of the first and second elongated members are connected to the four or more pedicle screws.

6. The cross connector of claim 1, wherein the four or more pedicle screws includes first, second, third, and fourth pedicle screws, and wherein the plurality of connecting devices include:
a first connecting device connecting the first end of the first elongated member to the first pedicle screw,
a second connecting device connecting the second end of the first elongated member to the second pedicle screw,
a third connecting device connecting the first end of the second elongated member to the third pedicle screw, and
a fourth connecting device connecting the second end of the second elongated member to the fourth pedicle screw.

7. The cross connector of claim 1, wherein the plurality of connecting devices include four or more articulated rods, each of the four or more articulated rods includes:
a rod segment configured to be received and secured by one of the four or more pedicle screws, and
a joint coupled to the rod and configured to engage one of the first end or the second end of the one of the first elongated member or the second elongated member.

8. The cross connector of claim 1, wherein the four or more pedicle screws includes first, second, third, and fourth pedicle screws, and wherein the plurality of connecting devices include two extendable devices, each having:
an adjustable rod having an adjustable length, the adjustable rod configured to be received and secured by at least two of the four or more pedicle screws, such that the adjustable rod is anchored to the spinal bone segments, and
a pair of joints coupled to both ends of the adjustable rod, the pair of joints configured to engage the first end of the first elongated member and the second end of the second elongated member, or the first end of the second elongated member and the second end of the first elongated member, such that adjustable length of the adjustable rod is configured to increase or decrease depending on the relative movement between the first and second elongated members.

9. The cross connector of claim 1, wherein the fulcrum member is formed along the pivot segment of the first elongated member, the fulcrum member includes:
top and bottom sections defining a substantially cylindrical space therebetween,
a pair of side walls coupled to the first and second ends of the first elongated member, the pair of side walls formed between the top and bottom sections and defining first and second ports for receiving the pivot segment of the second elongated member, and
a pivot member formed within the cylindrical space and configured to engage the pivot segment of the second elongated member.

10. The cross connector of claim 1, wherein the pivot segment of the first elongated member defines a first pivot port, the pivot segment of the second elongated member defines a second pivot port, and the fulcrum member includes:
top and bottom plates, and
a pivot pole coupled to the top and bottom plates and configured to engage the pivot segments of first and second elongated members by penetrating the first and second pivot ports.

11. A cross connector for use in conjunction with first and second stabilizing rods for stabilizing and protecting one or more fixation levels of spinal bone segments, the first and second stabilizing rods configured to be anchored to left and right pedicles of the spinal bone segments, the cross connector configured to be anchored to the spinal bone segments via the first and second stabilizing rods, the cross connector comprising:
first and second elongated members each having first and second ends and a pivot segment positioned between the first and second ends;
a fulcrum member configured to engage the pivot segment of the first elongated member and the pivot segment of the second elongated member, thereby allowing a relative movement therebetween;
a first anchoring device anchoring the first end of the first elongated member to the first stabilizing rod;
a second anchoring device anchoring the second end of the first elongated member to the second stabilizing rod;
a third anchoring device anchoring the first end of the second elongated member to the second stabilizing rod; and
a fourth anchoring device anchoring the second end of the second elongated member to the first stabilizing rod, such that the first and second elongated members are configured to form an X-shape bridge across the one or more fixation levels of spinal bone segments.

12. The cross connector of claim 11, wherein the X-shape bridge is configured to arch over the one or more fixation levels of spinal bone segments.

13. The cross connector of claim 12, wherein each of the first and second elongated members has:
an arched section along the pivot segment, and
first and second flat sections along the first and second ends.

14. The cross connector of claim 11, wherein the first and second elongated members each has an adjustable length.

15. The cross connector of claim 11, wherein the relative movement between the first and second elongated members is substantially restricted after the first and second ends of the first and second elongated members are anchored to the first and second stabilizing rods.

16. The cross connector of claim 11, wherein each of the first, second, third, and fourth anchoring devices has:
a hook member configured to engage one of the first and second stabilizing rods, and
a locking screw engaging the hook member and one of the first end or the second end of one of the first elongated member or the second elongated member, the locking screw configured to secure one of the first stabilizing rod or the second stabilizing rod to the respective hook member.

17. The cross connector of claim 11, wherein the fulcrum member is formed along the pivot segment of the first elongated member, the fulcrum member includes:
top and bottom sections defining a substantially cylindrical space therebetween,
a pair of side walls coupled to the first and second ends of the first elongated member, the pair of side walls formed between the top and bottom sections and defining first and second ports for receiving the pivot segment of the second elongated member, and
a pivot member formed within the cylindrical space and configured to engage the pivot segment of the second elongated member.

18. The cross connector of claim 11, wherein the pivot segment of the first elongated member defines a first pivot port, the pivot segment of the second elongated member defines a second pivot port, and the fulcrum member includes:
top and bottom plates, and
a pivot pole coupled to the top and bottom plates and configured to engage the pivot segments of first and second elongated members by penetrating the first and second pivot ports.

19. A cross connector for use in conjunction with first and second stabilizing rods for stabilizing and protecting one or more fixation levels of spinal bone segments, the first and second stabilizing rods configured to be anchored to left and right pedicles of the spinal bone segments, the cross connector configured to be anchored to the spinal bone segments via the first and second stabilizing rods, the cross connector comprising:
a first arm configured to be anchored to the first stabilizing rod;
a center member having first and second ends and a pair of brackets joining the first and second ends to form a protection ring, the first end coupled to the first arm, the protection ring configured to laterally surround a spinous process of one of the spinal bone segment; and
a second arm coupled to the second end of the center member and configured to be anchored to the second stabilizing rod.

20. The cross connector of claim 19, wherein each of the pair of brackets has an adjustable length.

21. The cross connector of claim 19, wherein the first and second arms form an arch with the center member over one of the spinal bone segments.

22. The cross connector of claim 19, wherein the protection ring has a shape selected from a group consisting of square, rectangle, circle, oval, triangle, and combinations thereof.

23. The cross connector of claim 19, wherein the protection ring has a shape adaptive to a contour of a base section of the spinous process.

24. The cross connector of claim 19, further comprising:
a first anchoring device anchoring the first arm to the first stabilizing rod; and
a second anchoring device anchoring the second arm to the second stabilizing rod.

25. The cross connector of claim 24, wherein each of the first and second anchoring devices include:
a hook member configured to engage one of the first and second stabilizing rods, and
a locking screw engaging the hook member and one of the first and second arms, the locking screw configured to secure one of the first and second stabilizing rods to the respective hook member.

26. A cross connector comprising:
a ring member having a circumferential surface;
first and second arms, each of the first and second arms having first and second ends, the first ends of the first and second arms configured to be coupled to the circumferential surface of the ring member, such that the first and second arms form a first arched bridge for supporting the ring member; and
first and second connecting devices, the first connecting device configured to be coupled to the second end of the first arm, the second connecting device configured to be coupled to the second end of the second arm.

27. The cross connector of claim 26, wherein each of the first and second connecting devices comprising:
a lockable joint coupled to the second end of one of the respective first arm or the second arm, and
a hook member coupled to the lockable joint and configured to engage a stabilizing rod, wherein the hook member is configured to move about the lockable joint when the lockable joint is unlocked, and wherein the hook member is configured to be stationary in relative to the lockable joint when the lockable joint is locked.

28. The cross connector of claim 26, wherein each of the first and second connecting devices comprises:
a lockable joint coupled to the second end of one of the respective first arm or the second arm, and
a rod member coupled to the lockable joint and configured to be received by a pedicle screw, wherein the rod member is configured to move about the lockable joint when the lockable joint is unlocked, and wherein the rod member is configured to be stationary in relative to the lockable joint when the lockable joint is locked.

29. The cross connector of claim 26, further comprising:
a first lockable joint coupled between the first end of the first arm and the circumferential surface of the ring member, wherein the first arm is configured to move about the lockable joint when the first lockable joint is unlocked, and wherein the first arm is configured to be stationary in relative to the first lockable joint when the first lockable joint is locked; and
a second lockable joint coupled between the first end of the second arm and the circumferential surface of the ring member, wherein the second arm is configured to move about the second lockable joint when the second lockable joint is unlocked, and wherein the second arm is configured to be stationary in relative to the second lockable joint when the lockable joint is locked.

30. The cross connector of claim 26, further comprising:
third and fourth arms, each of the third and fourth arms having first and second ends, the first ends of the third and fourth arms configured to be coupled to the circumferential surface of the ring member, such that the third and fourth arms form a second arched bridge for further supporting the ring member, wherein the first and second arched bridge combine to form an X-shape arched bridge having a center opening defined by the ring member; and
third and fourth connecting devices, the third connecting device configured to be coupled to the second end of the third arm, the fourth connecting device configured to be coupled to the second end of the fourth arm.

31. The cross connector of claim 30, wherein each of the first, second, third, and fourth connecting devices comprises:
a lockable joint coupled to the second end of one of the respective first arm, the second arm, third arm, or fourth arm, and
a hook member coupled to the lockable joint and configured to engage a stabilizing rod, wherein the hook member is configured to move about the lockable joint when the lockable joint is unlocked, and wherein the hook member is configured to be stationary in relative to the lockable joint when the lockable joint is locked.

32. The cross connector of claim 30, wherein each of the first, second, third, and fourth connecting devices comprises:
a lockable joint coupled to the second end of one of the respective first arm, the second arm, the third arm, or the fourth arm, and
a rod member coupled to the lockable joint and configured to be received by a pedicle screw, wherein the rod member is configured to move about the lockable joint when the lockable joint is unlocked, and wherein the rod member is configured to be stationary in relative to the lockable joint when the lockable joint is locked.

33. The cross connector of claim 30, further comprising:
a first lockable joint coupled between the first end of the first arm and the circumferential surface of the ring member;
a second lockable joint coupled between the first end of the second arm and the circumferential surface of the ring member;
a third lockable joint coupled between the first end of the third arm and the, circumferential surface of the ring member; and
a fourth lockable joint coupled between the first end of the fourth arm and the circumferential surface of the ring member;
wherein the first, second, third, and fourth arms are configured to move about the respective first, second, third, and fourth lockable joints when the respective first, second, third, and fourth lockable joints are unlocked, and wherein the first, second, third, and fourth arms are configured to be stationary in relative to the respective first, second, third, and fourth lockable joints when the respective first, second, third, and fourth lockable joints are locked.

34. A lockable joint for coupling a connecting device to an end of a cross connector, the lockable joint comprising:
a housing having a top surface, a side wall, an inner socket surface, a top receiving port formed on the top surface, an a side receiving port formed on the side wall, the side wall configured to be coupled to the connecting device;
a bearing disposed within the housing and contacting the inner socket surface of the housing;
a handle coupled to the bearing, the handle configured to extend outside the housing via the side receiving port, and configured to be coupled to the end of the cross connector, the handle having a range of multi-axle movement about the side receiving port; and
a locking screw having a concave surface, the locking screw configured to engage the housing via the top receiving port, the concave surface configured to apply a compression force against the bearing when the locking screw is at a locking position, the compression force substantially restricting the range of multi-axle movement of the handle.
